(19) United States
(12) Patent Application Publication
(10) Pub. No.: US 2008/0167649 A1
(43) Pub. Date: Jul. 10, 2008

(54) ABLATION APPARATUS AND METHOD

(75) Inventors: Stuart D. Edwards, Los Altos, CA (US); James Baker, Palo Alto, CA (US); Hugh Sharkey, Redwood Shores, CA (US); Ronald G. Lax, Grass Valley, CA (US)

Correspondence Address: ANGIODYNAMICS, INC., 603 QUEENSBURY AVENUE, QUEENSBURY, NY, 12804, US

(73) Assignee: AngioDynamics, Inc., Queensbury, NY (US)

(21) Appl. No.: 12/051,361

(22) Filed: Mar. 19, 2008

Related U.S. Application Data
(63) Continuation of application No. 11/034,503, filed on Jan. 12, 2005, now abandoned.
Application No. 11/034,503 is a continuation of application No. 10/700,605, filed on Nov. 3, 2003, now Pat. No. 7,150,744.
Application No. 10/700,605 is a continuation of application No. 09/513,725, filed on Feb. 24, 2000, now Pat. No. 6,641,580.
Application No. 09/513,725 is a continuation-in-part of application No. 09/383,166, filed on Aug. 25, 1999, now Pat. No. 6,471,698.
Application No. 09/383,166 is a continuation of application No. 08/802,195, filed on Feb. 14, 1997, now Pat. No. 6,071,280.
Application No. 08/802,195 is a continuation-in-part of application No. 08/515,379, filed on Aug. 15, 1995, now Pat. No. 5,683,384.
Application No. 08/515,379 is a continuation-in-part of application No. 08/290,031, filed on Aug. 12, 1994, now Pat. No. 5,536,267.
Application No. 09/513,725 is a continuation-in-part of application No. 09/364,203, filed on Jul. 30, 1999, now Pat. No. 6,663,624.
Application No. 09/364,203 is a continuation of application No. 08/623,652, filed on Mar. 29, 1996, now Pat. No. 5,935,123.
Application No. 08/623,652 is a division of application No. 08/295,166, filed on Aug. 24, 1994, now Pat. No. 5,599,345.

(51) Int. Cl. A61B 18/18

(52) U.S. Cl. 606/41; 606/33

(57) ABSTRACT

A delivery device is disclosed, which may include a needle and at least one deployable electrode retractable into the needle in a retracted geometry that may be substantially straight. The at least one deployable electrode may be operatively connectable to a radiofrequency energy source for delivery of radiofrequency energy. At least a distal portion of the at least one electrode may be deployable, from the needle in a lateral direction relative to a longitudinal axis of the needle, to a deployed geometry that may include at least one radius of curvature in three planes. The deployed geometry may include a helical portion.

### CROSS-REFERENCE TO RELATED APPLICATIONS

This application is a continuation of U.S. application Ser. No. 11/034,503, filed Jan. 12, 2005, now pending, which is a continuation of Ser. No. 10/700,605, filed Nov. 3, 2003, now U.S. Pat. No. 7,150,744, which is a continuation of U.S. application Ser. No. 09/513,725, filed Feb. 24, 2000, now U.S. Pat. No. 6,641,580, which is a continuation-in-part of U.S. application Ser. No. 09/383,166, filed Aug. 25, 1999, now U.S. Pat. No. 6,471,698, which is continuation of U.S. application Ser. No. 08/802,195, filed Feb. 14, 1997, now U.S. Pat. No. 6,071,280, which is a continuation-in-part of U.S. application Ser. No. 08/515,379, filed Aug. 15, 1995, now U.S. Pat. No. 5,683,384, which is a continuation-in-part of U.S. application Ser. No. 08/290,031, filed Aug. 12, 1994, now U.S. Pat. No. 5,536,267; the Ser. No. 09/513,725 application is also a continuation-in-part of U.S. application Ser. No. 09/364,203, filed Jul. 30, 1999, now U.S. Pat. No. 6,663,624, which is a continuation of U.S. application Ser. No. 08/623,652, filed Mar. 29, 1996, now U.S. Pat. No. 5,935,123, which is a divisional of U.S. application Ser. No. 08/295,166, filed Aug. 24, 1994, now U.S. Pat. No. 5,599,345; all of these related applications are incorporated in their entirety by express reference thereto.

### FIELD OF THE ART

This application relates generally to an apparatus for the treatment and ablation of body masses, such as tumors, and more particularly, to an RF treatment system suitable for treatment with retractable needle electrode.

Current open procedures for treatment of tumors are extremely disruptive and cause a great deal of damage to healthy tissue. During the surgical procedure, the physician must exercise care in not cutting the tumor in a manner that creates seeding of the tumor, resulting in metastasis. In recent years development of products has been directed with an emphasis on minimizing the traumatic nature of traditional surgical procedures.

There has been a relatively significant amount of activity in the area of hyperthermia as a tool for treatment of tumors. It is known that elevating the temperature of tumors is helpful in the treatment and management of cancerous tissues. The mechanisms of selective cancer cell eradication by hyperthermia are not completely understood. However, four cellular effects of hyperthermia on cancerous tissue have been proposed, (i) changes in cell or nuclear membrane permeability or fluidity, (ii) cytoplasmic lysomal disintegration, causing release of digestive enzymes, (iii) protein thermal damage affecting cell respiration and the synthesis of DNA or RNA and (iv) potential excitation of immunologic systems. Treatment methods for applying heat to tumors include the use of direct contact radio-frequency (RF) applicators, microwave radiation, inductively coupled RF fields, ultrasound, and a variety of simple thermal conduction techniques.

### SUMMARY

The present application describes an elongated delivery device having a lumen and an infusion array positionable in the lumen. The infusion array includes an RF electrode and at least two infusion members. Each infusion member has a tissue piercing distal portion and an infusion lumen. The infusion members are positionable in the elongated delivery device in a compacted state and deployable from the elongated delivery device with curvature in a deployed state. Also, the infusion members exhibit a changing direction of travel when advanced from the elongated delivery device to a selected tissue site.

In another embodiment, an electrode is deployably positioned at least partially in a delivery catheter. The electrode is in a non-deployed state when positioned within the delivery catheter. As it is advanced out the distal end of the catheter the electrode becomes deployed. The electrode has a first section with a first radius of curvature, and a second section, extending beyond the first section, having a second radius of curvature or a substantially linear geometry. Alternatively, the electrode has at least two radii of curvature that are formed when advanced through the delivery catheter's distal end. The deployed electrode can have at least one radius of curvature in two or more planes.

In another embodiment, a delivery device may include a needle and at least one deployable electrode retractable into the needle in a retracted geometry that may be substantially straight. The at least one deployable electrode may be operatively connectable to a radiofrequency energy source for delivery of radiofrequency energy. At least a distal portion of the at least one electrode may be deployable, from the needle in a lateral direction relative to a longitudinal axis of the needle, to a deployed geometry that may include at least one radius of curvature in three planes. The deployed geometry may include a helical portion, such as the one shown in FIG. 4. The needle may further include an insulation sleeve and an exposed distal portion. The distal portion of the needle may further include a thermal sensor. The at least one electrode may include three electrodes, each including at least one radius of curvature in three planes. The distal portion of the at least one electrode may be deployable from a distal end of the needle.

In another embodiment, a delivery device may include a means for puncturing through skin or percutaneous entry, and at least one electrode retractable into the puncturing means in a retracted geometry. The at least one electrode may be operatively connectable to a radiofrequency energy source. At least a distal portion of the at least one electrode may be deployable from the puncturing means to a deployed geometry that may include at least one radius of curvature in two or more planes. The distal portion of the at least one electrode may be deployable from the puncturing means in a lateral direction relative to a longitudinal axis of the puncturing means. The distal portion of the at least one electrode may be deployable from a distal end of, or a side opening along, the puncturing means. The retracted geometry may be substantially straight. The deployed geometry may include a helical portion. The puncturing means may be an insert, an introducer, a needle, or an electrode. The delivery device may further include a handle coupled to the puncturing means. The puncturing means may include an insulation sleeve and an exposed distal portion. The distal portion of the puncturing means may include a thermal sensor, such as a thermocouple.

In another embodiment, a method of delivery is disclosed herein, which involves providing a delivery device that includes a skin puncturing means and at least one electrode retractable in a substantially straight geometry within the puncturing means, and deploying at least a distal portion of the at least one electrode from the puncturing means so that the deployed distal portion includes at least one radius of curvature in two or more planes. The at least one electrode may be operatively connectable to a radiofrequency energy source. The method may further include deploying the distal portion of the at least one electrode in a lateral direction relative to a longitudinal axis of the puncturing means. The method may further include providing a thermal sensor on a distal portion of the puncturing means for measuring tissue temperature.

### BRIEF DESCRIPTION OF THE DRAWINGS

FIG. 1 is a perspective view of a tissue treatment apparatus according to the present application, including a delivery catheter, a handle, and deployed electrodes.

FIG. 2 is a cross-sectional view of the tissue treatment apparatus illustrated in FIG. 1.

FIG. 3 is a perspective view of three deployed electrodes according to the present application, each including two radii of curvature.

FIG. 4 is a perspective view of a deployed electrode according to the present application, including one radius of curvature in three planes.

FIG. 5 is a perspective view of two deployed electrodes according to the present application.

FIG. 6 is a perspective view of two deployed electrodes according to the present application.

FIG. 7 is a cross-sectional view of a delivery catheter according to the present application, including a guide tube positioned at the distal end of the delivery catheter.

FIG. 8 is a perspective view of an electrode according to the present application.

FIG. 9 is a perspective view of the tissue treatment apparatus shown in FIG. 1, with the delivery catheter being introduced percutaneously through the body and positioned at the exterior, or slightly piercing, a liver with a tumor.

FIG. 10 is a perspective view of a tissue treatment apparatus according to the present application, including an obturator positioned in the delivery catheter.

FIG. 11 is a perspective view of the tissue treatment apparatus shown in FIG. 10, positioned in the body adjacent to the liver, with the obturator removed.

FIG. 12 is a perspective view of the tissue treatment apparatus shown in FIG. 10, positioned in the body adjacent to the liver, and the electrode deployment apparatus, with an electrode template, is positioned in the delivery catheter in place of the obturator.

FIG. 13 is a perspective view of a tissue treatment apparatus according to the present application, with deployed electrodes surrounding a tumor.

FIG. 14 is a perspective view of a tissue treatment apparatus according to the present application, positioned in the body adjacent to the liver, with deployed electrodes surrounding a tumor and infusing a solution to the tumor site during a pre-ablation procedure.

FIG. 15 is a perspective view of a tissue treatment apparatus according to the present application, illustrating application of RF energy to the tumor.

FIG. 16 is a perspective view of a tissue treatment apparatus according to the present application, illustrating the electro-desiccation of the tumor.

FIG. 17 is a perspective view of a tissue treatment apparatus according to the present application, illustrating the instillation of solutions to the tumor site during a post-ablation procedure.

FIG. 18 illustrates bipolar ablation between electrodes according to the present application.

FIG. 19 illustrates monopolar ablation using electrodes according to the present application.

FIG. 20 is a perspective view of a tissue treatment system according to the present application, including RF and ultrasound modules, and a monitor.

FIG. 21 is a block diagram of components in a tissue treatment system according to the present application.

FIG. 22A is a cross-sectional view of a treatment apparatus according to the present application.

FIG. 22B is a cross-sectional view of the distal end of a treatment apparatus according to the present application.

FIG. 22C is a cross-sectional view of a treatment apparatus according to the present application, illustrating the proximal end of an insulation sleeve and a thermocouple associated with the insulation sleeve.

FIG. 22D is a close up cross-sectional view of a treatment apparatus according to the present application, illustrating the proximal end of the treatment apparatus.

FIG. 23 is an exploded view of a treatment apparatus according to the present application.

FIG. 24 is a partial cross-sectional view of a treatment apparatus according to the present application, illustrating an electrode, an insulation sleeve, and the associated thermal sensors.

FIG. 25A is a perspective view of a treatment apparatus according to the present application, including an infusion device mounted at the distal end of a catheter.

FIG. 25B is a perspective view of the treatment apparatus of FIG. 25A, illustrating the removal of the catheter, and an electrode attached to the distal end of the catheter, from the infusion device which is left remaining in the body.

FIG. 26A is a perspective view of a treatment apparatus according to the present application, including an electrode mounted at the distal end of the catheter.

FIG. 26B is a perspective view of the treatment apparatus of FIG. 26A, illustrating the removal of an introducer from the lumen of the electrode.

FIG. 27A is a perspective view of the treatment apparatus of FIG. 26B, with the introducer removed from the lumen of the electrode.

FIG. 27B is a perspective view of the treatment apparatus of FIG. 27A, illustrating the removal of the electrode from the catheter, leaving behind the insulation sleeve.

FIG. 28A is a perspective view of a treatment apparatus according to the present application, including an insulation sleeve positioned in a surrounding relationship to an electrode, which is mounted to the distal end of a catheter.

FIG. 28B is a perspective view of the treatment apparatus of FIG. 28A, illustrating the removal of the insulation sleeve from the electrode.

FIG. 28C is a perspective view of the insulation sleeve of FIG. 28B, after it is removed from the electrode.

FIG. 29A is a perspective view illustrating the attachment of a syringe.

FIG. 29B is a perspective view of a syringe, containing a fluid medium such as a chemotherapeutic agent, attached to the treatment apparatus of FIG. 27A.

FIG. 30 is a block diagram of components in an energy source of a treatment system according to the present application.

FIGS. 31A-1 and 31A-2 are panels of a schematic diagram of a power supply.

FIG. 31B is a schematic diagram of a voltage sensor.

FIG. 31C is a schematic diagram of a current sensor.

FIG. 31D is a schematic diagram of a power computing circuit.

FIG. 31E is a schematic diagram of an impedance computing circuit.

FIG. 31F is a schematic diagram of a power control device.

FIGS. 31G-1 through 31G-4 are panels of a schematic diagram of an eight-channel temperature measurement circuit.

FIGS. 31H-1 and 31H-2 are panels of a schematic diagram of a power and temperature control circuit.

FIG. 32 is a block diagram of an embodiment according to the present application, which includes a microprocessor.

FIG. 33 illustrates the use of two electrodes configured to operate in a bipolar mode.

FIG. 34 is a perspective view of an embodiment of a tissue treatment apparatus according to the present application, illustrating a delivery catheter coupled to a power source.

FIG. 35 is a perspective view of a treatment apparatus according to the present application, illustrating a primary electrode and a single laterally deployed secondary electrode.

FIG. 36 is a perspective view of a conic ablation volume achieved with the apparatus of FIG. 35.

FIG. 37 is a perspective view of a treatment apparatus according to the present application, which includes two secondary electrodes.

FIG. 38 is a perspective view illustrating the adjacent positioning of the apparatus of FIG. 37 next to a tumor.

FIG. 39 is a perspective view illustrating the positioning of the apparatus of FIG. 37 in a tumor, and the creation of a cylindrical ablation volume.

FIG. 40A is a perspective view of a treatment apparatus according to the present application that includes two secondary electrodes which provide a retaining and gripping function.

FIG. 40B is a perspective view of a treatment apparatus according to the present application that includes three secondary electrodes which provide a retaining and gripping function.

FIG. 40C is a cross-sectional view of the apparatus of FIG. 40B taken along the lines 6C-6C.

FIG. 41 is a perspective view of a treatment apparatus according to the present application that includes three secondary electrodes deployable from a distal end of an insulation sleeve surrounding a primary electrode.

FIG. 42 is a perspective view of a treatment apparatus according to the present application that includes two secondary electrodes deployable from a primary electrode, and three secondary electrodes deployable from the distal end of an insulation sleeve surrounding the primary electrode.

FIG. 43 is a block diagram illustrating a control system that includes a controller, an energy source, and other electronic components according to the present application.

FIG. 44 is a block diagram illustrating the inclusion of an analog amplifier, an analog multiplexer, and a microprocessor according to the present application.

### DETAILED DESCRIPTION

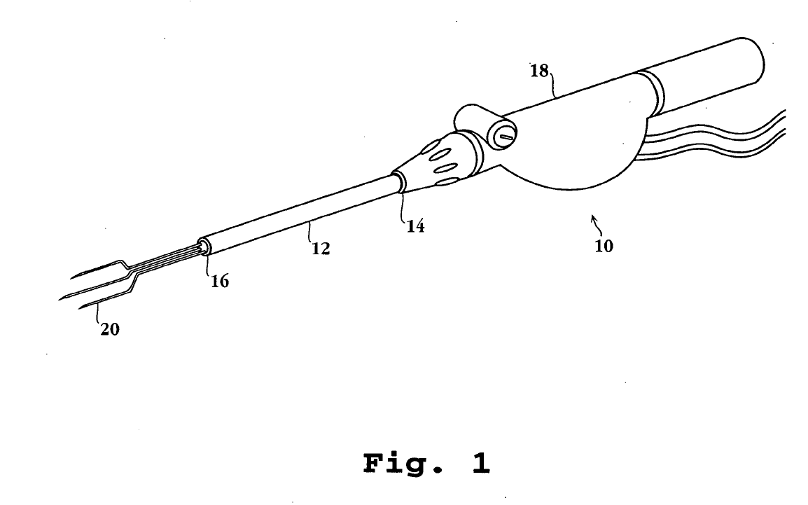

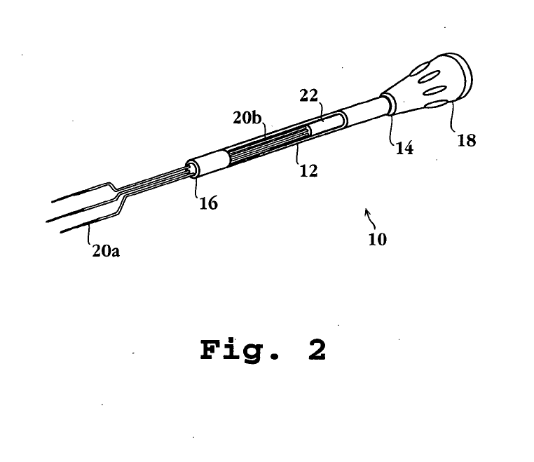

A tissue treatment apparatus 10 according to the present application is illustrated in FIG. 1. Treatment apparatus 10 includes a delivery catheter 12 with a proximal end 14 and a distal end 16. Delivery catheter 12 can be of the size of about 5 French to 16 French. A handle 18 may be removably attached to proximal end 14. An electrode deployment device is at least partially positioned within delivery catheter 12, and includes a plurality of electrodes 20 that are retractable into and deployable out of distal end 16. Electrodes 20 can be of different sizes, shapes and configurations. In one embodiment, they are needle electrodes, with sizes in the range of 27 gauge to 14 gauge. Electrodes 20 are in non-deployed positions while retained in delivery catheter 12. In the non-deployed positions, electrodes 20 may be in a compacted state, spring loaded, generally confined or substantially straight. As electrodes 20 are advanced out of distal end 16 they become distended in a deployed state, which defines an ablative volume, in which tissue is ablated as illustrated more fully in FIG. 2. Electrodes 20 operate either in the bipolar or monopolar modes. When the electrodes 20 are used in the bipolar mode, the ablative volume is substantially defined by the peripheries of the plurality of electrodes 20. In one embodiment, the cross-sectional width of the ablative volume is about 4 cm. However, it will be appreciated that different ablative volumes can be achieved with tissue treatment apparatus 10.

The ablative volume is first determined to define a mass, such as a tumor, to be ablated. Electrodes 20 may be placed in a surrounding relationship to a mass or tumor in a predetermined pattern for volumetric ablation. An imaging system is used to first define the volume of the tumor or selected mass. Suitable imaging systems include but are not limited to, ultrasound, computerized tomography (CT) scanning, X-ray film, X-ray fluoroscopy, magnetic resonance imaging, electromagnetic imaging, and the like. The use of such devices to define a volume of a tissue mass or a tumor is well known to those skilled in the art.

With regard to the use of ultrasound, an ultrasound transducer transmits ultrasound energy into a region of interest in a patient's body. The ultrasound energy is reflected by different organs and different tissue types. Reflected energy is sensed by the transducer, and the resulting electrical signal is processed to provide an image of the region of interest. In this way, the ablation volume is then ascertained.

The ablative volume is substantially defined before treatment apparatus 10 is introduced to an ablative treatment position. This assists in the appropriate positioning of treatment apparatus 10. In this manner, the volume of ablated tissue is reduced and substantially limited to a defined mass or tumor, optionally including a certain area surrounding such a tumor that is well controlled and defined. A small area around the tumor may be ablated in order to ensure that the entire tumor is ablated.

With reference again to FIG. 2, electrode sections 20a are in deployed states when they are introduced out of distal end 16. Although electrodes 20 are generally in a non-distended configuration in the non-deployed state while positioned in delivery catheter 12, they can also be distended. Generally, electrode sections 20b are in retained positions while they are non-deployed. This is achieved by a variety of methods including but not limited to: (i) the electrodes are pre-sprung, confined in delivery catheter 12, and only become sprung (expanded) as they are released from delivery catheter 12, (ii) the electrodes are made of a memory metal, as explained in further detail below, (iii) the electrodes are made of a selectable electrode material which gives them an expanded shape outside of delivery catheter 12, or (iv) delivery catheter 12 includes guide tubes which serve to confine electrodes 12 within delivery catheter 12 and guide their direction of travel outside of the catheter to form the desired, expanded configurations. As shown in FIG. 2, electrodes 20 are pre-sprung while retained in delivery catheter 12. This is the non-deployed position. As they are advanced out of delivery catheter 12 and into tissue, electrodes 20 become deployed and begin to “fan” out from distal end 16, moving in a lateral direction relative to a longitudinal axis of delivery catheter 12. As deployed electrodes 20 continue their advancement, the area of the electrode “fan” increases and extends beyond the diameter of distal end 16.

Each electrode 20 is distended in a deployed position, and collectively, the deployed electrodes 20 define a volume of tissue that will be ablated. As previously mentioned, when ablating a tumor, either benign or malignant, one may ablate an area that is slightly in excess to that defined by the exterior surface of the tumor. This improves the chances that the entire tumor is eradicated.

Deployed electrodes 20 can have a variety of different deployed geometries including but not limited to, (i) a first section with a first radius of curvature, and a second section, extending beyond the first section, having a second radius of curvature or a substantially linear geometry, (ii) at least two radii of curvature, (iii) at least one radius of curvature in two or more planes, (iv) a curved section, with an elbow, that is located near distal end 16 of delivery catheter, and a non-curved section that extends beyond the curved section, or (v) a curved section near distal end 16, a first linear section, and then another curved section or a second linear section that is angled with regard to the first linear section. Deployed electrodes 20 need not be parallel with respect to each other. The plurality of deployed electrodes 20, which define a portion of the needle electrode deployment device, can all have the same deployed geometries, i.e., all with at least two radii of curvature, or a variety of geometries, i.e., one with two radii of curvature, a second one with one radius of curvature in two planes, and the rest a curved section near distal end 16 of delivery catheter 12 and a non-curved section beyond the curved section.

A cam 22, or other actuating device, can be positioned within delivery catheter and used to retract and advance electrodes 20 in and out of delivery catheter 12. The actual movement of the actuating device can be controlled at handle 18. Suitable cams are of conventional design, well known to those skilled in the art.

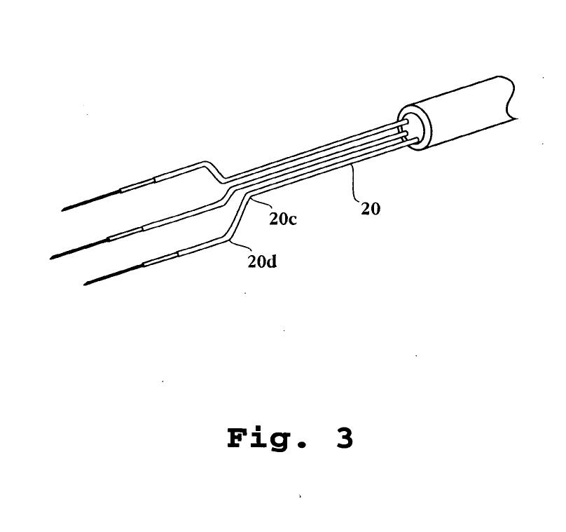

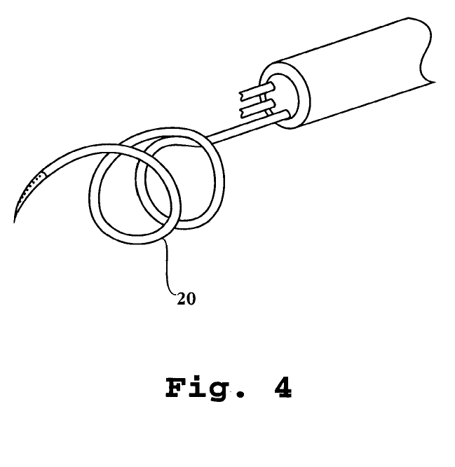

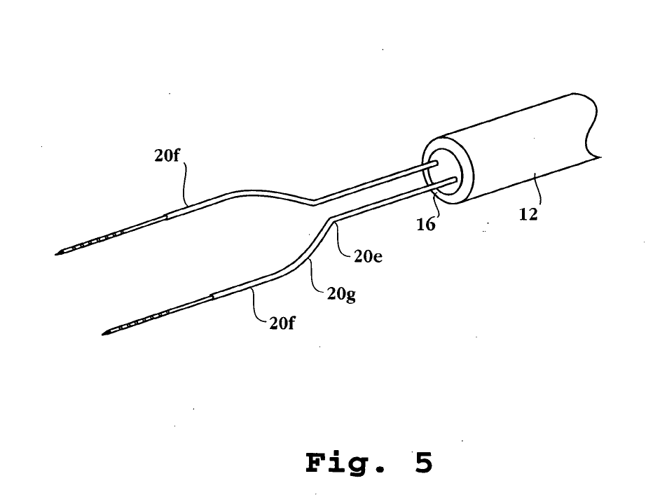

The different geometric configurations of electrodes 20 are illustrated in FIGS. 3 through 6. In FIG. 3, each of the three electrodes 20 has a first radius of curvature 20c and a second radius of curvature 20d. The electrode can include more than two radii of curvature. As shown in FIG. 4, electrode 20 has at least one radius of curvature which extends to three planes. In FIG. 5, each of the two electrodes has a curved section 20e which is near distal end 16 of delivery catheter 12. A generally linear section 20f extends beyond curved section 20e, and sections 20e and 20f meet at an elbow 20g. Each of the electrodes can serve as anode or cathode. The plurality of electrodes can have linear sections 20f that are generally parallel to each other, or sections 20f can be non-parallel. FIG. 6 illustrates two electrodes that each includes a curved section 20e positioned near distal end 16 of delivery catheter 12, a linear section 20f extending beyond curved section 20e, sections 20e and 20f meeting at an elbow 20g, and a section 20h which extends beyond linear section 20f. Section 20h can be linear, curved, or a combination of the two. The plurality of electrodes illustrated in FIG. 6 can have parallel or non-parallel linear sections 20f.

Suitable electrode materials include stainless steel, platinum, gold, silver, copper and other electromagnetic energy conducting materials including conductive polymers. In one embodiment, electrode is made of a memory metal, such as nickel titanium, commercially available from Raychem Corporation, Menlo Park, Calif. A resistive heating element can be positioned in an interior lumen of electrode. Resistive heating element can be made of a suitable metal that transfers heat. Not all of electrode needs to be made of a memory metal. It is possible that only a distal portion of electrode which is introduced into tissue be made of the memory metal. Mechanical devices, including but not limited to steering wires, can be attached to the distal portion of electrode to cause it to become directed, deflected and move about in a desired direction about the tissue, until it reaches its final resting position to ablate a tissue mass.

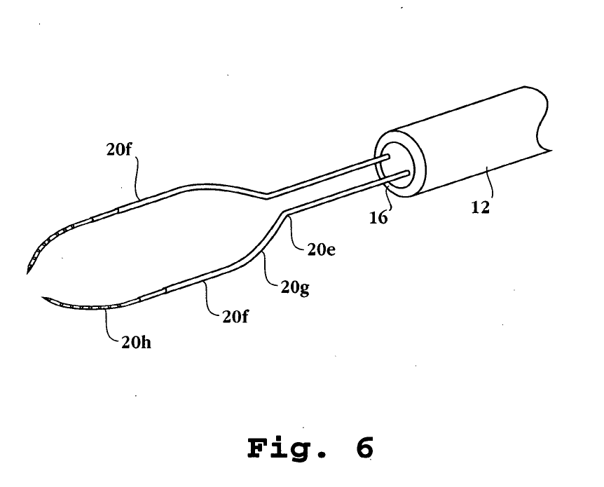

As shown in FIG. 7, optionally included in delivery catheter 12 are one or more guide tubes 24, which serve to direct the expansion of electrodes 20 as they are advanced out of distal end 16 of delivery catheter 12. Guide tubes 24 can be made of stainless steel, spring steel and thermal plastics, including but not limited to nylon and polyesters, and are of sufficient size and length to accommodate the electrodes 20 to a specific site in the body.

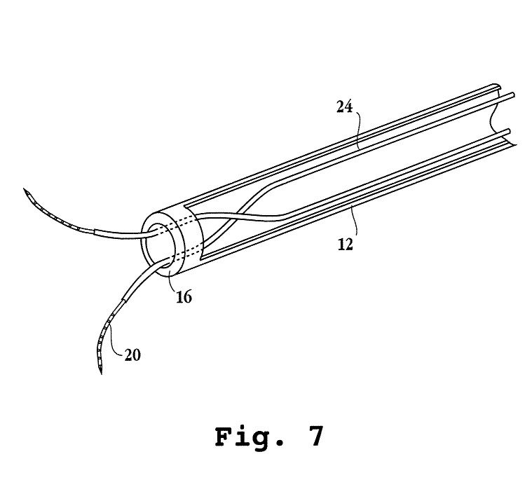

FIG. 8 illustrates one embodiment of electrode 20 with a sharpened distal end 25. By including a tapered, or piercing, distal end 25, the advancement of electrode 20 through tissue is facilitated. Electrode 20 can be segmented, and may include a plurality of fluid distribution ports 26, which can be evenly formed around all or only a portion of electrode 20. Fluid distribution ports 26 are formed in electrode 20 when it is hollow and permit the introduction and flow of a variety of fluidic mediums through electrode 20 to a desired tissue site. Such fluidic mediums include, but are not limited to, electrolytic solutions, pastes, gels, as well as chemotherapeutic agents. Examples of suitable conductive gels include carboxymethyl cellulose gels, optionally including aqueous electrolytic solutions such as physiological saline solutions, and the like.

The size of fluid distribution ports 26 can vary, depending on the size and shape of electrode 20. Also associated with electrode 20 is an adjustable insulation sleeve 28 that is slidable along an exterior surface of electrode 20. Insulation sleeve 28 may be advanced and retracted along electrode 20 in order to define the size of a conductive surface of electrode 20. Insulation sleeve 28 is actuated at handle 18 by the physician, and its position along electrode 20 is controlled. When electrode 20 is deployed out of delivery catheter 12 and into tissue, insulation sleeve 28 can be positioned around electrode 20 as it moves its way through the tissue. Alternatively, insulation sleeve 28 can be adjusted along electrode 20 to provide a desired length of conductive surface after electrode 20 has been positioned relative to a targeted mass to be ablated. Insulation sleeve is thus capable of advancing through tissue along with electrode 20, or it can move through tissue without electrode 20 providing the source of movement. The desired ablation volume is defined by deployed electrodes 20, optionally in combination with the positioning of insulation sleeve 28 on each electrode. In this manner, a very precise ablation volume is created. Suitable materials that form insulation sleeve include but are not limited to nylon, polyimides, other thermoplastics, and the like.

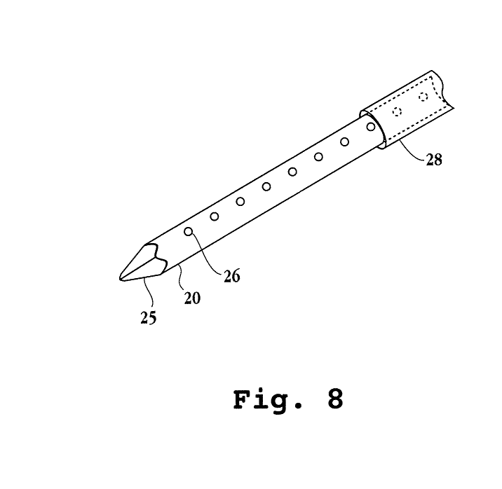

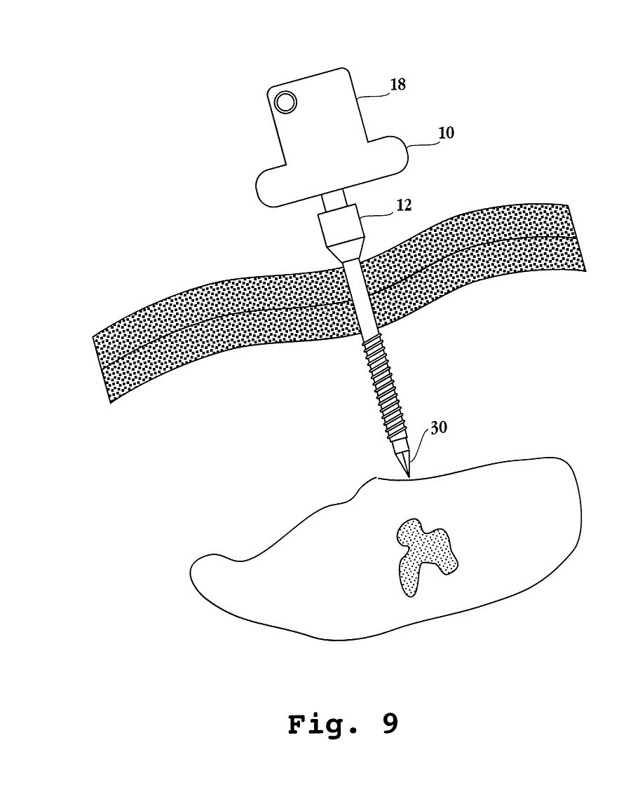

FIG. 9 illustrates a percutaneous application of tissue treatment apparatus 10. Tissue treatment apparatus 10 can be used percutaneously to introduce electrodes to the selected tissue mass or tumor. Electrodes 20 can remain in their non-deployed positions while being introduced percutaneously into the body, and delivered to a selected organ which contains the selected mass to be ablated. Delivery catheter 12 is removable from handle 18. Electrode deployment device (including the plurality of electrodes) can be inserted into and removed from delivery catheter 12. An obturator 30 may be inserted into delivery catheter 12 initially to facilitate a percutaneous procedure. As shown in FIG. 10, obturator 30 can have a sharpened distal end 32 that pierces tissue and assists the introduction of delivery catheter 12 to a selected tissue site. The selected tissue site can be a body organ (e.g., liver) with a tumor or other mass therein, or the actual tumor itself.

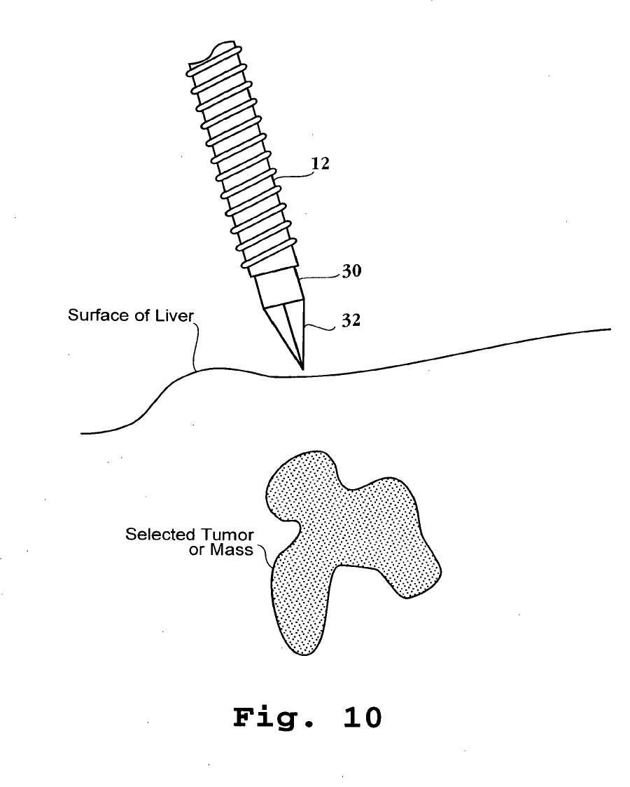

As shown in FIG. 11, obturator 30 may then be removed from delivery catheter 12. As shown in FIG. 12, electrode deployment device (including the deployable electrodes) is then inserted into delivery catheter 12, and the catheter 12 is then reattached to handle 18. Electrode deployment device can optionally include an electrode template 34 to guide the deployment of electrodes 20. The electrodes may be deployed to a surrounding relationship at an exterior of a selected mass in the tissue.

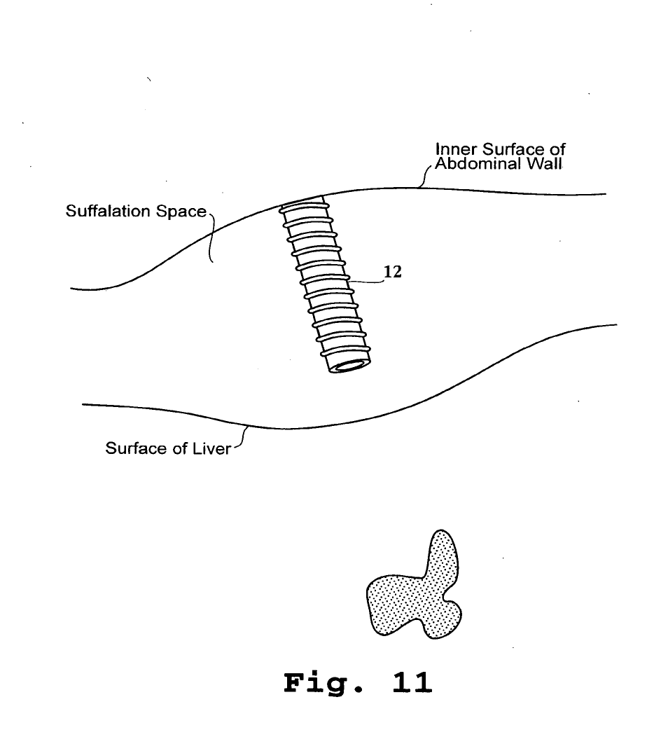

As shown in FIG. 13, electrodes 20 are then advanced out of distal end 16 of delivery catheter 12, and become deployed to form a desired ablative volume. Each individual electrode 20 pierces the liver and travels therethrough until being positioned relative to the tumor. The ablative volume is selectable, and determined first by imaging the area to be ablated. The ablative volume is defined by the peripheries of all of the deployed electrodes 20. Once the volume of ablation is determined, then an electrode set is selected which will become deployed to define the ablation volume. Different electrodes 20 may have various degrees of deployment, based on type of electrode material, the level of pre-springing of the electrodes and the geometric configuration of the electrodes in their deployed states. Tissue treatment apparatus 10 permits different electrode sets to be deployed from delivery catheter 12 in order to define a variety of ablative volumes.

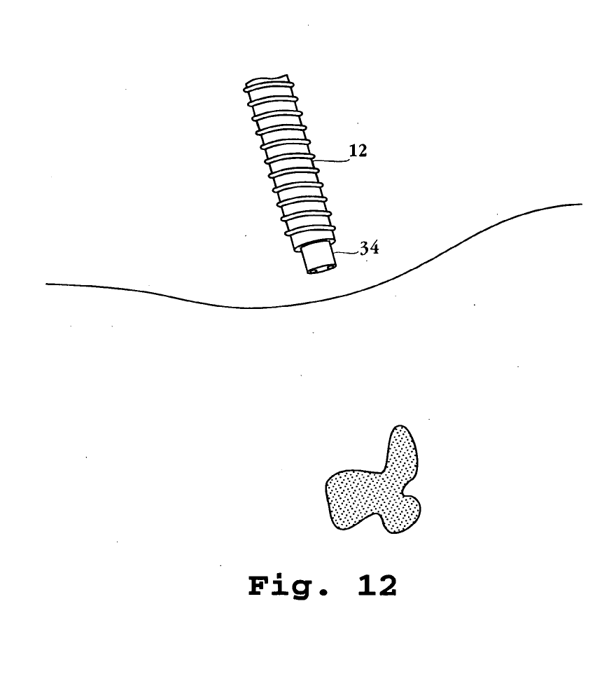

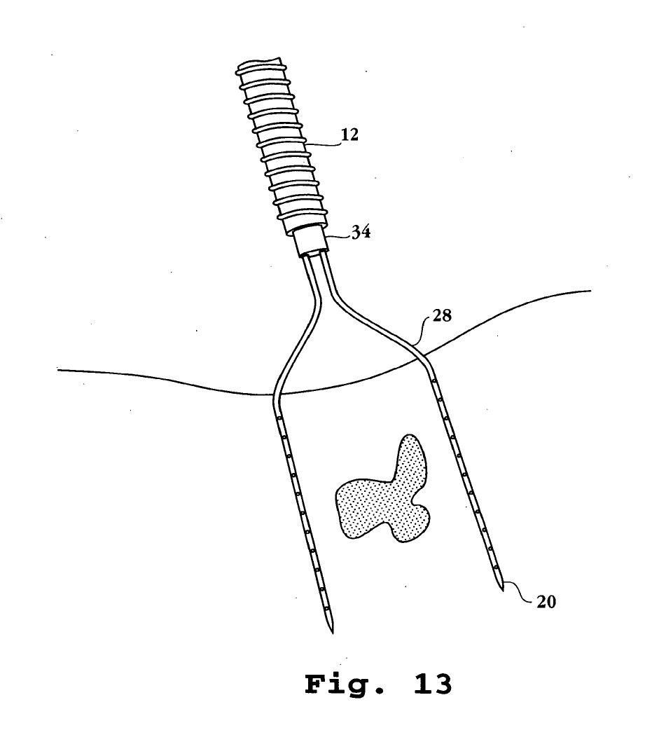

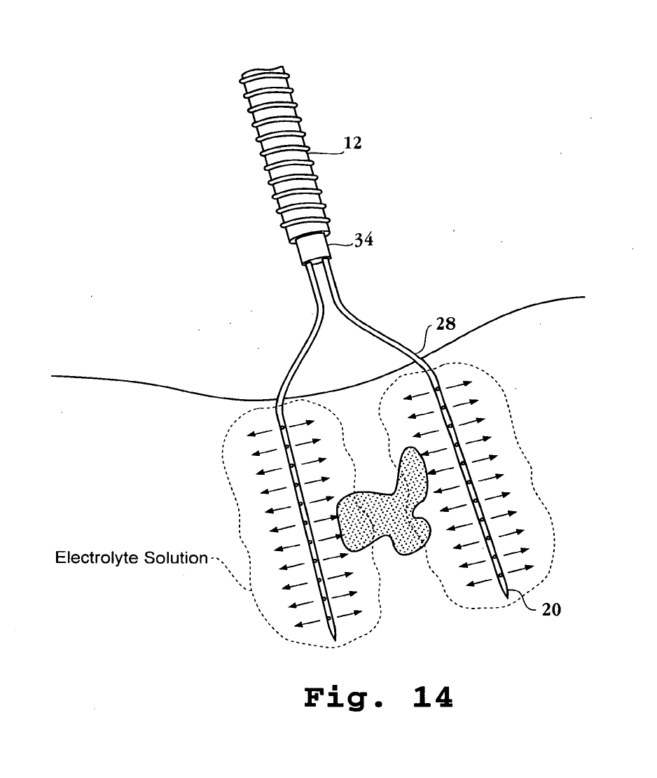

Prior to ablating the tumor, a pre-ablation step can be performed. A variety of different solutions, including electrolytic solutions such as saline, can be introduced through deployed electrodes 20 to the tumor site, as shown in FIG. 14. FIG. 15 illustrates the application of RF energy to ablate the tumor. Electrode insulator 28 is positioned on portions of electrodes 20 where there will be no energy delivery for ablation. The positioning of insulator 28 along electrode 20 may further define the ablation volume. The actual electro-desiccation of the tumor, or other targeted masses or tissues, is shown in FIG. 16. Again, deployed electrodes 20, with their electrode insulators 28 positioned along sections of the electrodes 20, define the ablation volume, and the resulting amount of mass that is desiccated. Optionally, following desiccation, electrodes 20 can be used to introduce a variety of solutions to the ablated tissue in a post-ablation process. This step is illustrated in FIG. 17. Suitable solutions include, but are not limited to, pharmacological agents, chemotherapeutic agents.

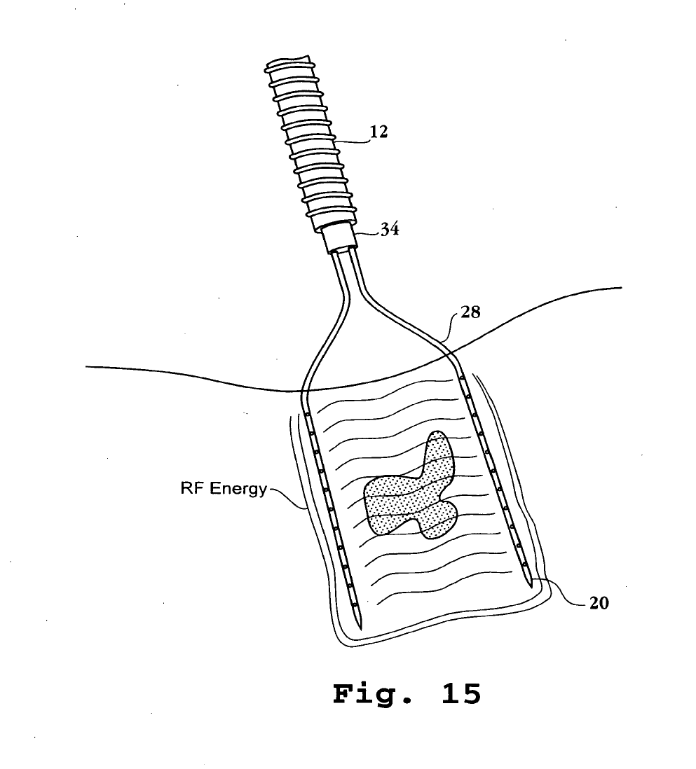

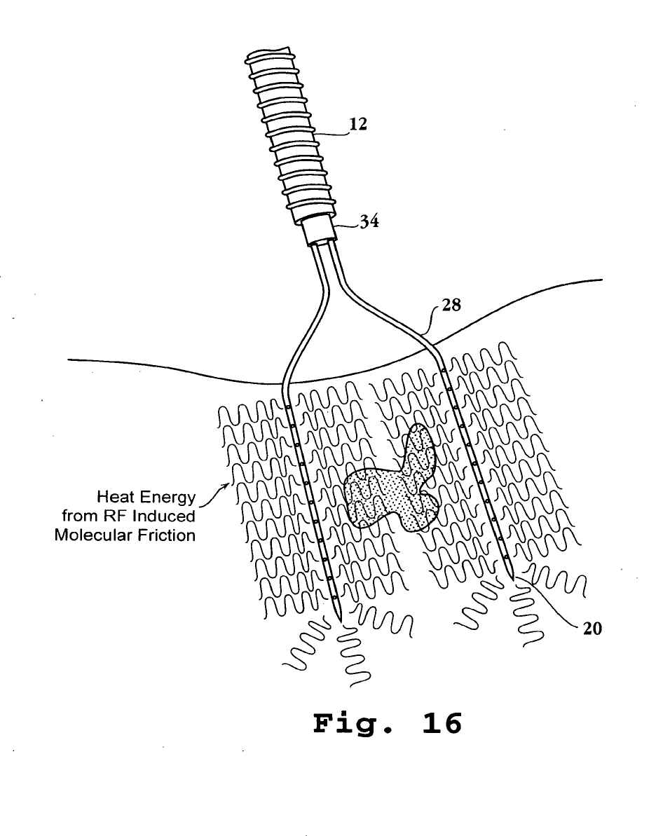

FIG. 18 illustrates a tissue treatment apparatus having two electrodes 20 operating in a bipolar mode in delivering energy to a selected tissue. FIG. 19 illustrates a tissue treatment apparatus having two electrodes 20 operating in a monopolar mode in delivering energy to a selected tissue. Each of the plurality of electrodes 20 in a tissue treatment apparatus can operate in different mode (e.g., bipolar or monopolar) in the ablation process. Electrical polarity may be shifted between the different electrodes.

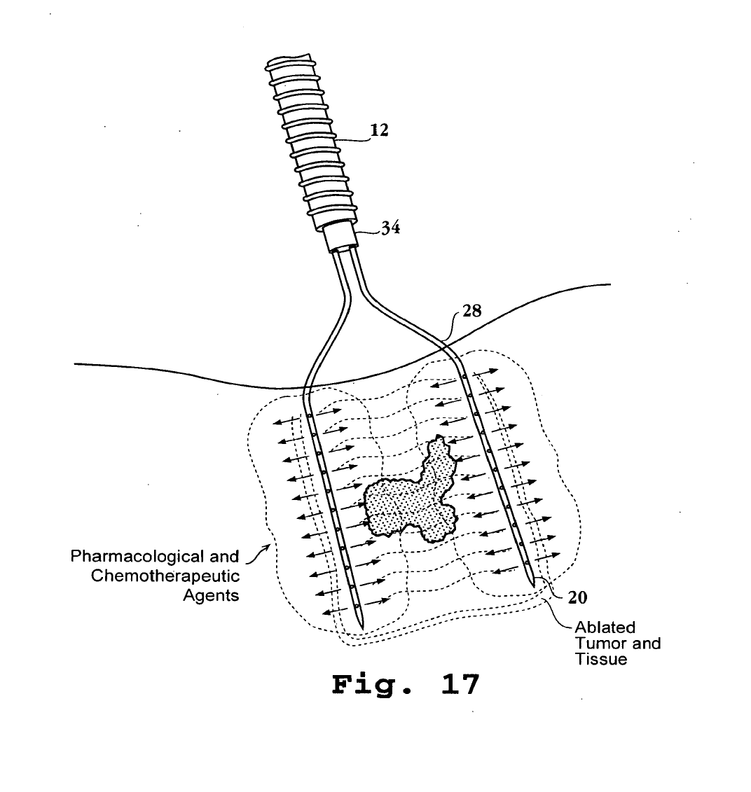

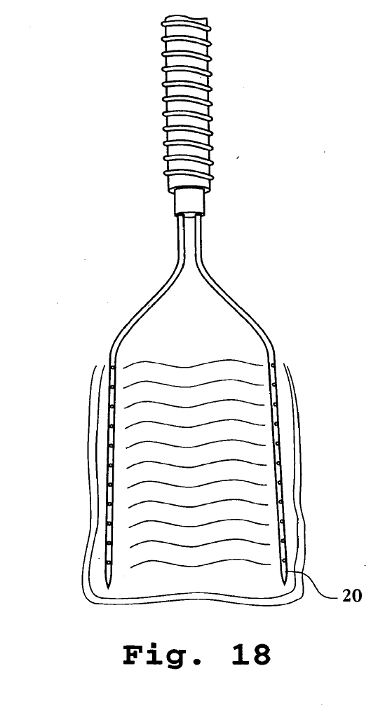

FIG. 20 shows a tissue treatment system 36, which can be modular. Tissue treatment system 36 can include one or more of a display 38, an RF energy source, a microwave source, an ultrasound source, a visualization device (such as cameras and VCRs), a source of fluidic medium (e.g., electrolytic solutions, pharmacological solutions, chemotherapeutic solutions, pastes, gels), and a controller which can be used to monitor temperature or impedance. An embodiment of a tissue treatment system that includes certain components listed herein is illustrated in FIG. 21. One of the deployed electrodes 20 can be a microwave antenna coupled to a microwave source. This electrode can initially be coupled to an RF energy source and is then switched to the microwave source.

Referring now to FIG. 21, a power supply 40 powers an electromagnetic energy source (e.g., RF generator) 42. An RF generator provides RF energy to electrodes of tissue treatment apparatus 10. A multiplexer 46 enables the measurements of current, voltage and temperature (at thermal sensors which can be positioned on electrodes). Multiplexer 46 is driven by a controller 48, which can be a digital or analog controller, or a computer with software. When controller 48 is a computer, it can include a CPU coupled through a system bus. The system may further include or be coupled to a keyboard, a disk drive, or other non-volatile memory systems, a display, and other peripherals, as known in the art. Also coupled to the bus may include a program memory and a data memory. Controller 48 is coupled to an operator interface 50, which includes operator controls 52 and display 38. Controller 48 is coupled to an imaging system, which may include ultrasound components (e.g., ultrasound transducers) or optical components (e.g., viewing optics, optical fibers).

Current and voltage measurements are used to calculate impedance. Tissue imaging may be carried out using ultrasound, CT scanning, or other methods known in the art. Imaging can be performed before, during and after treatment. The output of current sensors, voltage sensors, and thermal sensors is used by controller 48 to control the delivery of energy through electrodes of tissue treatment apparatus 10. Controller 48 can also control temperature of tissue about the electrodes, and power output at the electrodes. The amount of energy delivered controls the amount of power output at the electrodes. A profile of power delivered can be incorporated in controller 48. A pre-set amount of energy to be delivered can also be profiled. Feedback signals can include the measurements of impedance or temperature, and be processed at controller 48. Controller 48 may be incorporated in electromagnetic energy source 42. Tissue impedance can be calculated by supplying a small amount of non-ablation energy to electrodes of tissue treatment apparatus 10 and measuring voltage and current.

Circuitry, software and feedback to controller 48 result in process control and are used to: (i) change energy output at electrodes of tissue treatment apparatus 10, such as RF or microwave, (ii) change the duty cycle (on-off and wattage) of energy delivery, (iii) change mode of energy delivery (e.g., monopolar or bipolar), (iv) change fluidic medium delivery (e.g., flow rate and pressure), and (v) determine when ablation is complete. through, temperature and/or impedance measurements. These process variables can be controlled and varied based on time, temperature measurements taken at multiple sites, and/or impedance measurements (to current flow through tissue, indicating changes in current carrying capability of the tissue) during the ablative process.

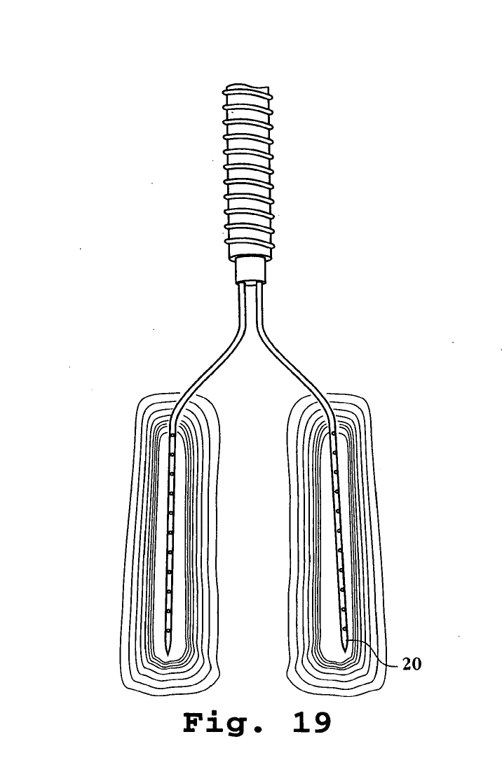

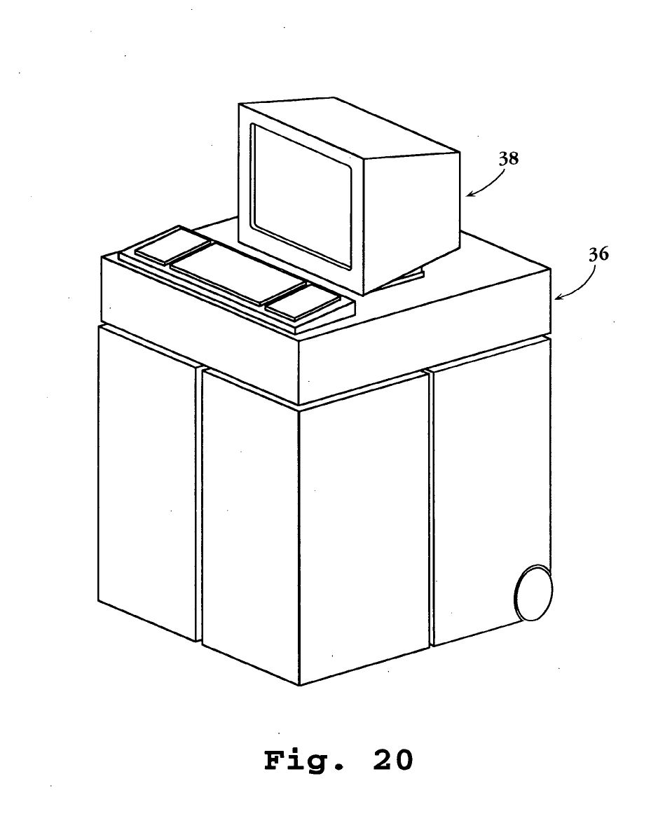

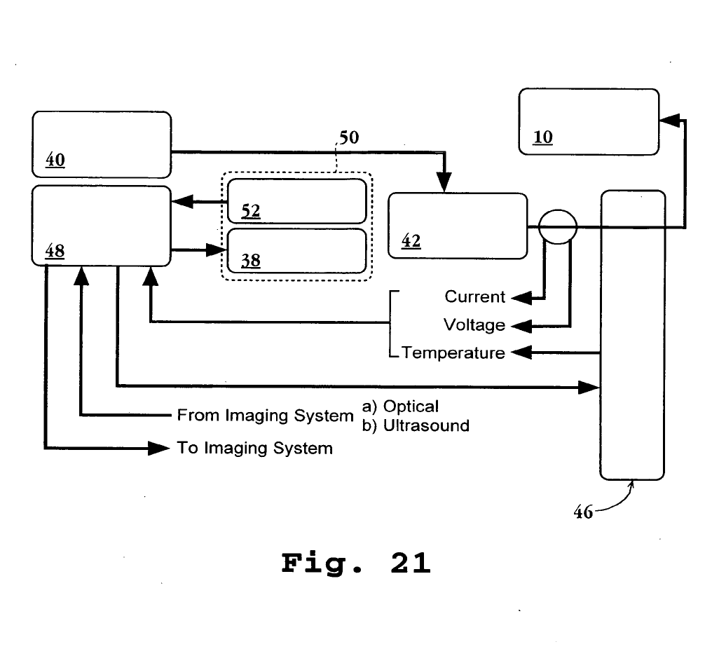

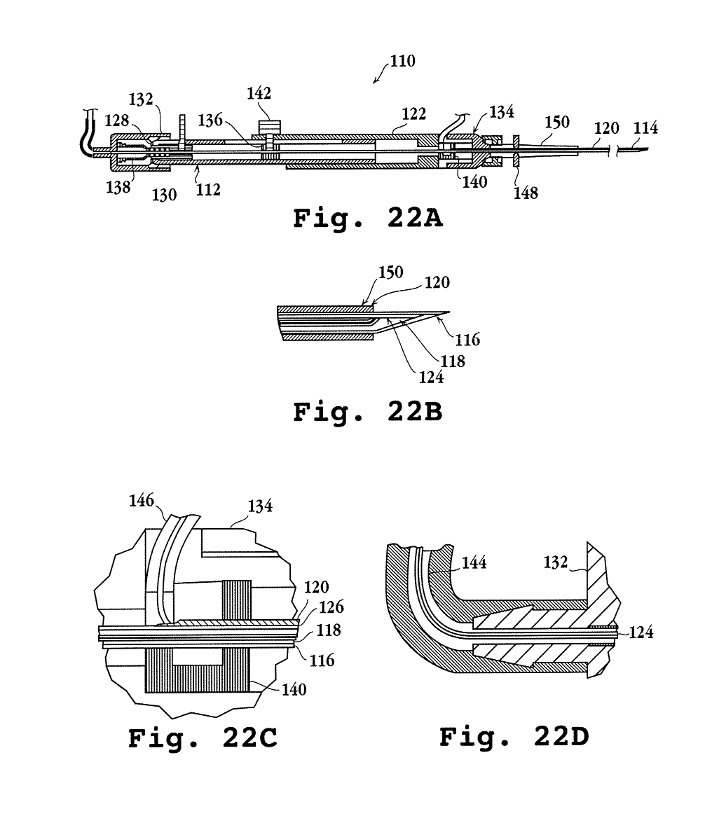

Referring now to FIG. 22A, a treatment apparatus 110 is illustrated which can be used to ablate a selected tissue mass, including but not limited to a tumor, by hyperthermia. Treatment apparatus 110 includes a catheter 112 with a catheter lumen in which different devices may be introduced and removed. An insert 114 is removably positioned in the catheter lumen. Insert 114 can be an introducer, a needle electrode, and the like.

When insert 114 is an introducer, including but not limited to a guiding or delivery catheter, it is used as a means for puncturing the skin of the body, and advancing catheter 112 to a desired site. Alternatively, insert 114 can be both an introducer and an electrode adapted to receive current for tissue ablation and hyperthermia.

Referring to FIGS. 22A and 22B, if insert 114 is not an electrode, then a removable electrode 116 may be positioned in insert 114 either during or after treatment apparatus 110 has been introduced percutaneously to the desired tissue site. Electrode 116 has an electrode distal end that advances out of a distal end of insert 114. In this deployed position, energy is introduced to the tissue site along a conductive surface of electrode 116.

Electrode 116 can be included in treatment apparatus 110, and positioned within insert 114, while treatment apparatus 110 is being introduced to the desired tissue site. The distal end of electrode 116 can have substantially the same geometry as the distal end of insert 114 so that the two ends are essentially flush. Distal end of electrode 116, when positioned in insert 114 as it is introduced through the body, serves to block material from entering the lumen of insert 114. The distal end of electrode 116 essentially can provide a plug type of function.

Electrode 116 is then advanced out of a distal end of insert 114, and the length of an electrode conductive surface is defined, as explained further in this application. Electrode 116 can advance straight, laterally or in a curved manner out of distal end of insert 114. Ablative or hyperthermia treatment may be carried out when two electrodes 116 are positioned to effect bipolar treatment of the desired tissue site or tumor. Operating in a bipolar mode, selective ablation of the tumor is achieved. The delivery of energy is controlled and the power output at each electrode may be maintained independent of changes in voltage or current. Energy is delivered slowly at low power, permitting a wide area of even ablation. In one embodiment, an RF power output of 8 W to 14 W is delivered in a bipolar mode for 10 to 25 minutes to achieve an ablation area between electrodes 116 of about 2 cm to 6 cm. However, it will be appreciated that the present invention is suitable for treating, through hyperthermia or ablation, different sizes of tumors or masses. When electrodes 116 are operated in monopolar mode, a return electrode is attached to the patient's skin.

Treatment apparatus 110 can also include a removable introducer 118 which is positioned in the insert lumen instead of electrode 116. Introducer 118 has an introducer distal end that also serves as a plug, to minimize the entrance of material into the insert distal end as it advances through a body structure. Introducer 118 is initially included in treatment apparatus 110, and is housed in the lumen of insert 114, to assist the introduction of treatment apparatus 110 to the desired tissue site. Once treatment apparatus 110 is at the desired tissue site, then introducer 118 is removed from the insert lumen, and electrode 116 is substituted in its place. In this regard, introducer 118 and electrode 116 are removable relative to insert 114.

Also included in treatment apparatus 110 is an insulation sleeve 120 coupled to an insulator slide 122. Insulation sleeve 120 is positioned in a surrounding relationship to electrode 116. Insulator slide 122 imparts a slidable movement of the insulation sleeve 120 along a longitudinal axis of electrode 116 in order to define an electrode conductive surface that begins at an insulation sleeve distal end.

The distal end of treatment apparatus 110 is shown in FIG. 22B. Introducer 118 is positioned in the lumen of electrode 116, which can be surrounded by insulation sleeve 120, all of which are essentially placed in the lumen of infusion device 150. It will be appreciated, however, that in FIG. 22B an insert (e.g., insert 114 as described herein) can take the place of electrode 116, and an electrode (e.g., electrode 116 as described herein) can be substituted for introducer 118. As such, electrode 116 may be positioned in the lumen of insert 114.

Referring to FIGS. 22A-22C, a sensor 124 can be positioned in or on electrode 116 or introducer 118. A sensor 126 is positioned on insulation sleeve 120. In one embodiment, sensor 124 is located at the distal end of introducer 118, and sensor 126 is located at the distal end of insulation sleeve 120, at an interior wall which defines a lumen of insulation sleeve 120. Suitable thermal sensors include a T type thermocouple (e.g., copper-constantan), J type thermocouple, E type thermocouple, K type thermocouple, thermistors, fiber optics, resistive wires, IR detectors, and the like. It will be appreciated that sensors 124 and 126 need not be thermal sensors. Catheter 112, insert 114, electrode 116 and introducer 118 can be made of a variety of materials. In one embodiment, catheter 112 is black anodized aluminum, 0.5 inch in diameter, electrode 116 is made of stainless steel (e.g., 18 gauge needle), introducer 118 is made of stainless steel (e.g., 21 gauge needle), and insulation sleeve 120 is made of polyimide.

By monitoring temperature, energy delivery can be accelerated to a predetermined or desired level. Impedance is used to monitor voltage and current. The readings of sensors 124 and 126 are used to regulate voltage and current that is delivered to the tissue site. The output for these sensors is used by a controller, described further in this application, to control the delivery of energy to the tissue site. Resources, which can be hardware and/or software, are associated with an energy source, coupled to electrode 116. The resources are associated with sensors 124 and 126, as well as the energy source for maintaining a selected power output at electrode 116 independent of changes in voltage or current.

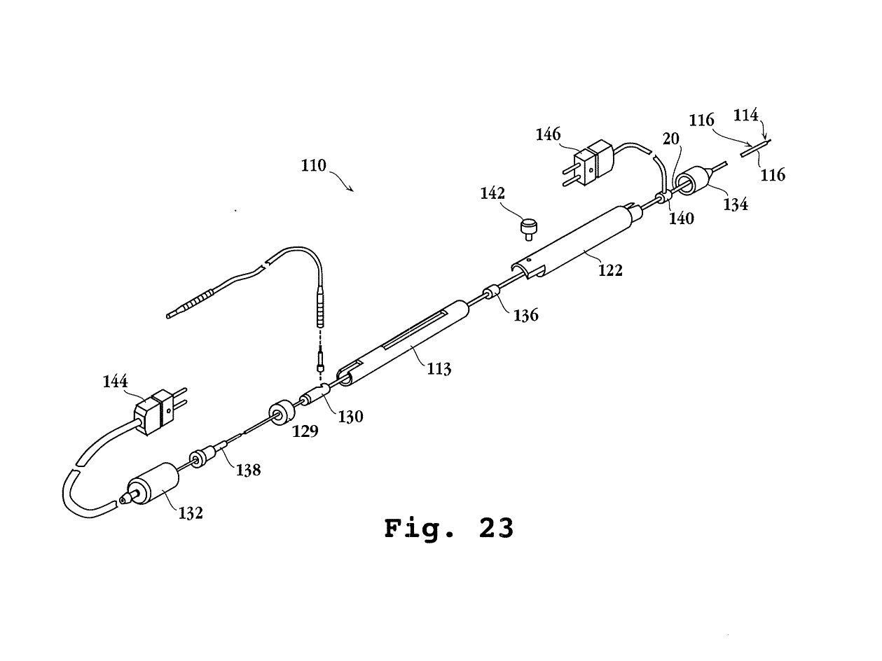

Referring to FIG. 24, electrode 116 may be hollow and include a plurality of fluid distribution ports 128 from which a variety of fluids can be introduced, including electrolytic solutions, chemotherapeutic agents, infusion media, and other fluidic media disclosed herein.

A specific embodiment of the treatment device 110 is illustrated in FIG. 23. Included is an electrode locking cap 129, an energy source coupler 130, an introducer locking cap 132, an insulator slide 122, a catheter body 113, an insulator retainer cap 134, an insulator locking sleeve 136, a luer connector 138, an insulator elbow connector 140, an insulator adjustment screw 142, a thermocouple cable 144 for thermal sensor 126, a thermocouple cable 46 for thermal sensor 124 and a luer retainer 148 for an infusion device 150.

In another embodiment of treatment apparatus 110, electrode 116 is directly attached to catheter 112 without insert 114. Introducer 118 is slidably positioned in the lumen of electrode 116. Insulation sleeve 120 is again positioned in a surrounding relationship to electrode 116 and is slidably moveable along its surface in order to define the conductive surface. Sensors 124 and 126 are positioned at the distal ends of introducer 118 and insulation sleeve 120. Alternatively, sensor 124 can be positioned on electrode 116, such as at its distal end. The distal ends of electrode 16 and introducer 118 can be sharpened and tapered. This assists in the introduction of treatment apparatus 110 to the desired tissue site. Each of the two distal ends of electrode 116 and introducer 118 can have geometries that essentially match. Additionally, distal end of introducer 118 can include an essentially solid end in order to prevent the introduction of material into the lumen of catheter 112.

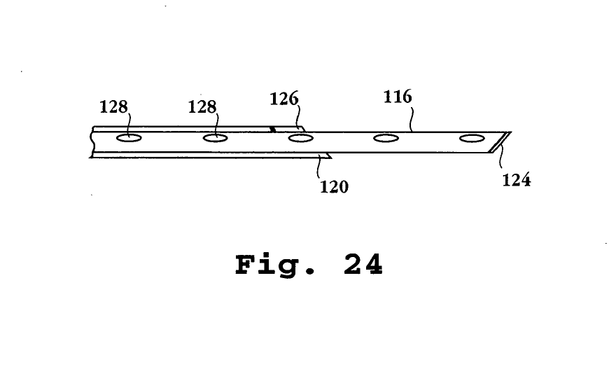

In yet another embodiment of treatment apparatus 110, as shown in FIGS. 25A and 25B, infusion device 150 is attached to the distal end of catheter 112 and retained by a collar. The collar is rotated, causing catheter 112 to become disengaged from infusion device 150. Electrode 116 is attached to the distal end of catheter 112. Infusion device 150 has an infusion device lumen and catheter 112 is at least partially positioned in the infusion device lumen. Electrode 116 is positioned in the catheter lumen, in a fixed relationship to catheter 112, but is removable from the lumen. Insulation sleeve 120 is slidably positioned along a longitudinal axis of electrode 116. Introducer 118 is positioned in a lumen of electrode 116 and is removable therefrom. An energy source is coupled to electrode 116. Resources are associated with sensors 124 and 126, and with voltage and current sensors that are coupled to the power source for maintaining a selected power output at electrode 116. Catheter 112 may be pulled away from infusion device 150, which also removes electrode 116 from infusion device 150. Thereafter, only infusion device 150 is retained in the body. This permits a chemotherapeutic agent, or other fluidic medium, to be easily introduced to treat the tissue site over an extended period of time. Additionally, by leaving infusion device 150 in place, electrode 116, introducer 118, and/or catheter 112 can be inserted through the lumen of infusion device 150 to the tissue site at a later time for additional treatment in the form of hyperthermia or ablation.

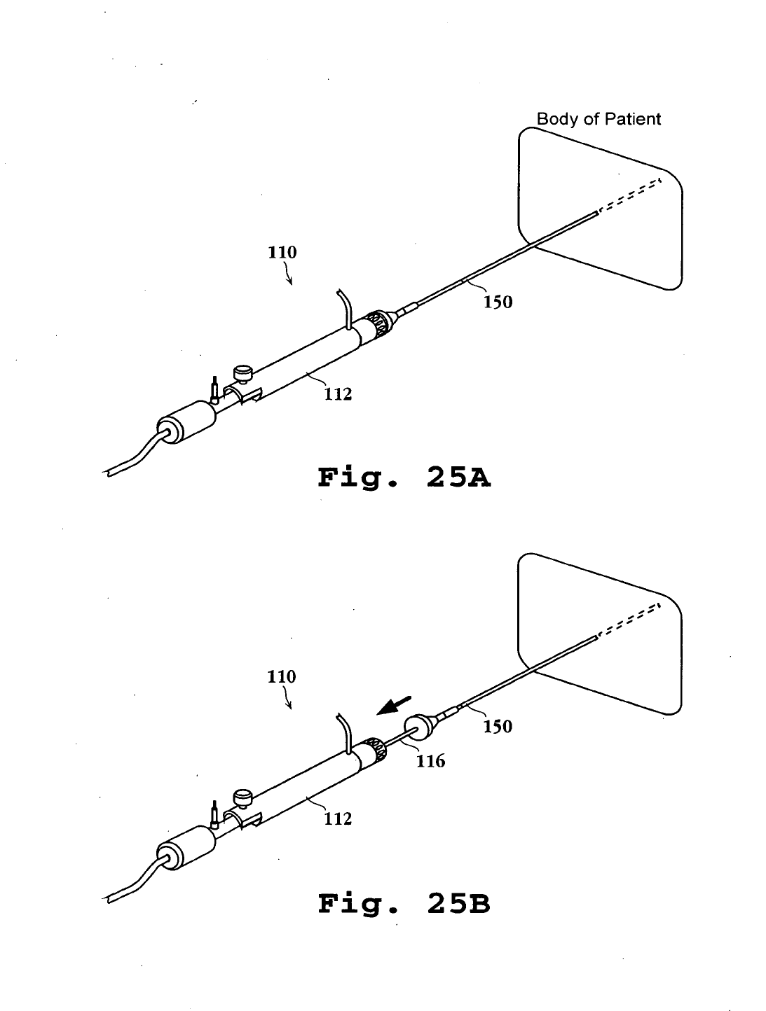

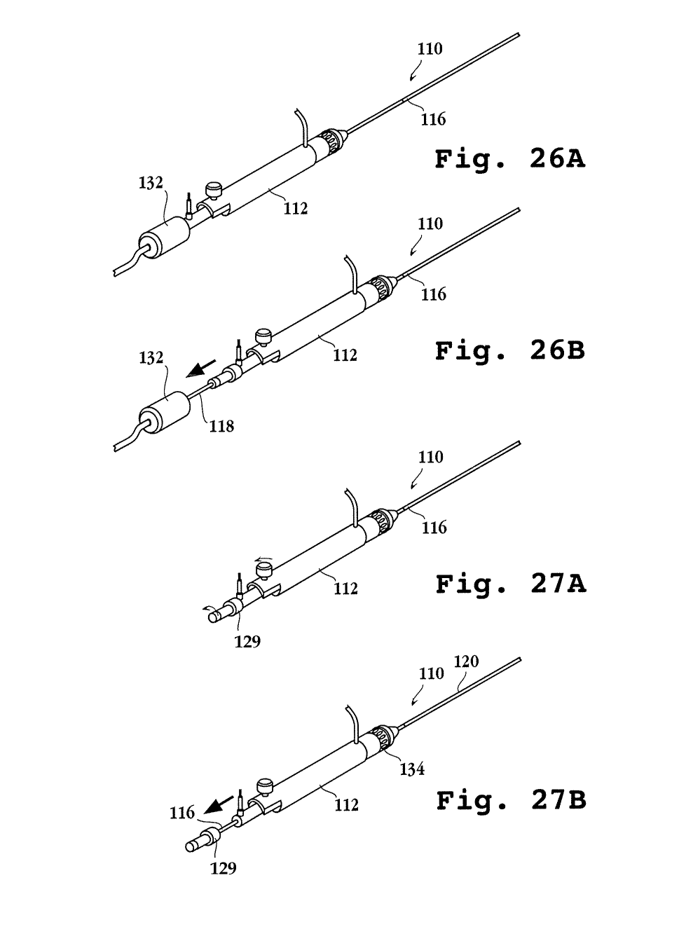

In FIG. 26A, electrode 116 is shown as attached to the distal end of catheter 112. Introducer 118 is attached to introducer locking cap 132 which is rotated and pulled away from catheter 112. As shown in FIG. 26B, this removes introducer 118 from the lumen of electrode 116.

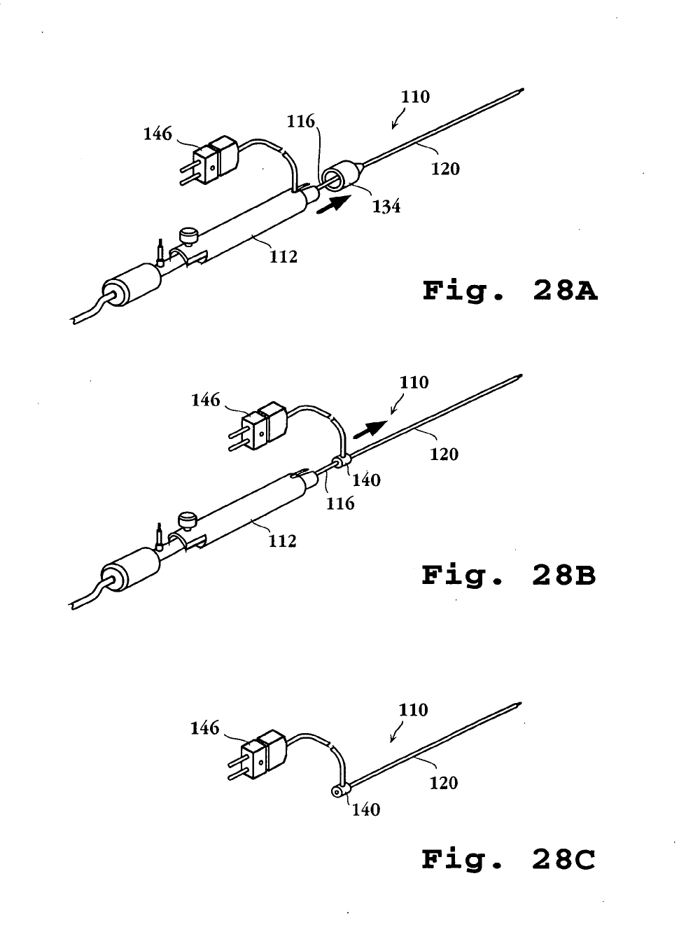

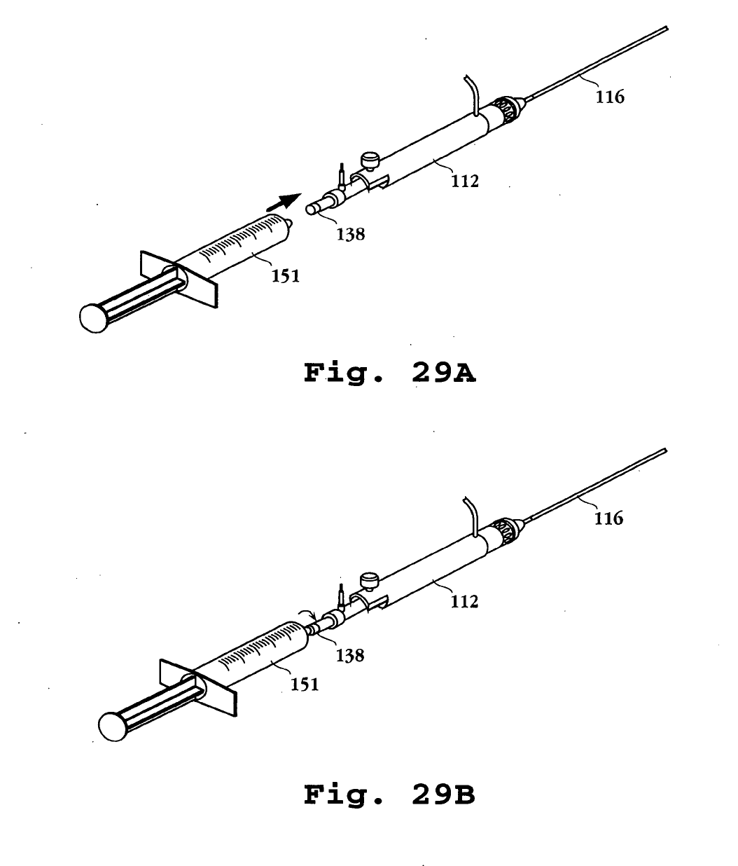

Referring now to FIG. 27A, electrode 116 is at least partially positioned in the lumen of catheter 112. Electrode locking cap 129 is mounted at the proximal end of catheter 112, with the proximal end of electrode 116 attached to electrode locking cap 129. Electrode locking cap 129 is rotated and to unlock electrode 116 from catheter 112. In FIG. 27B, electrode locking cap 129 is then pulled away from the proximal end of catheter 112, pulling with it electrode 116 which is then removed from the lumen of catheter 112. After electrode 116 is removed from catheter 112, insulation sleeve 120 is locked on catheter 112 by insulator retainer cap 134.

In FIG. 28A, insulator retainer cap 134 is unlocked and removed from catheter 112. As shown in FIG. 28B, insulation sleeve 120 is then slid off of electrode 116. FIG. 28C illustrates insulation sleeve 120 completely removed from catheter 112 and electrode 116.

Referring now to FIGS. 29A and 29B, after introducer 118 is removed from catheter 112, a fluid source, such as syringe 151 delivering a suitable fluidic medium, including but not limited to a chemotherapeutic agent, is attached to luer connector 138 at the proximal end of catheter 112. The fluidic medium is then delivered from syringe 151 through electrode 116 to the tumor site. Syringe 151 is then removed from catheter 112 by imparting a rotational movement of syringe 151 and pulling it away from catheter 112. Thereafter, electrode 116 can deliver further energy to the tumor site. Additionally, electrode 116 and catheter 112 can be removed, leaving only infusion device 150 in the body (see FIGS. 25A-25B). Syringe 151 can then be attached directly to infusion device 150 to introduce a fluidic medium to the tumor site. Alternatively, other fluid delivery devices can be coupled to infusion device 150 in order to have a more sustained supply of fluidic mediums to the tumor site. Once fluid delivery (e.g., chemotherapy) is completed, electrode 116 and catheter 112 can be introduced through infusion device 150. Energy is then delivered to the tumor site. The process begins again with the subsequent removal of catheter 112 and electrode 116 from infusion device 150. Fluid delivery (e.g., chemotherapy) can then begin again. Once fluid delivery is complete, further energy can be delivered to the tumor site by reintroducing electrode 116 through infusion device 150. This process can be repeated any number of times for an effective multi-modality treatment of the tumor site.

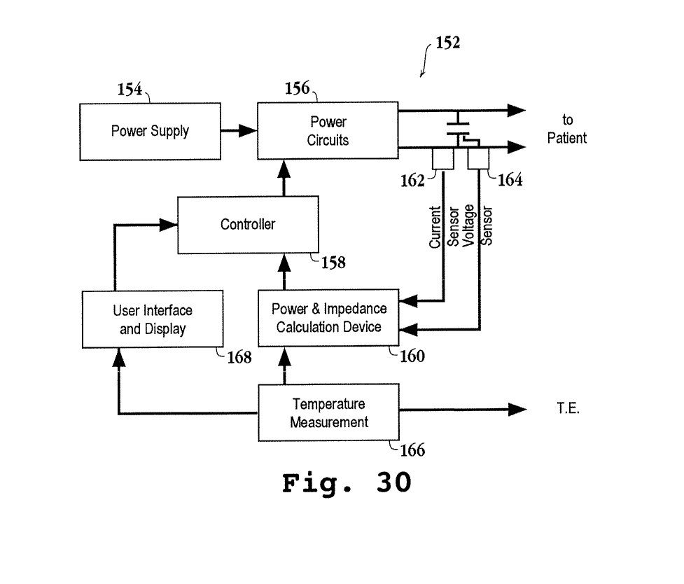

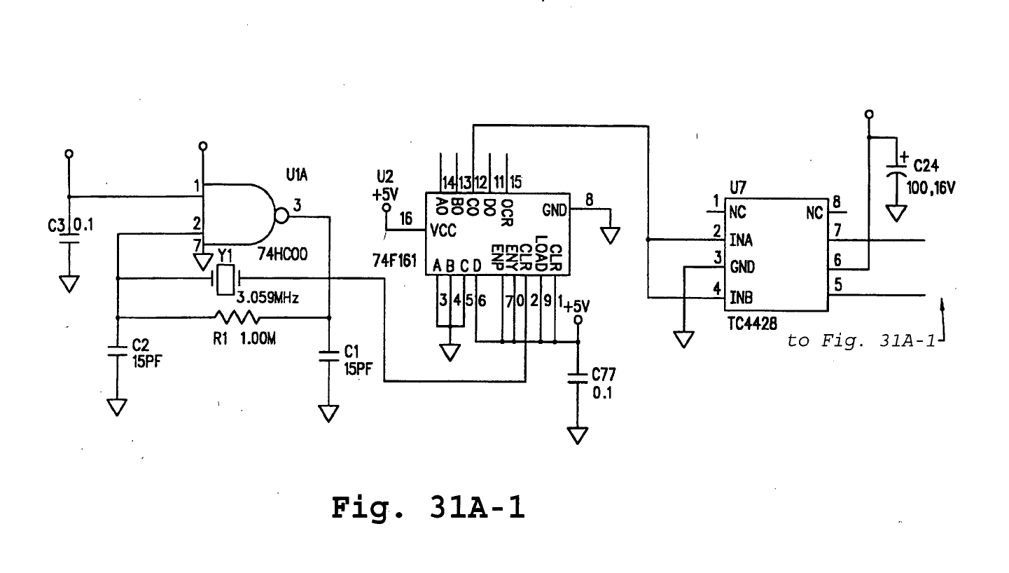

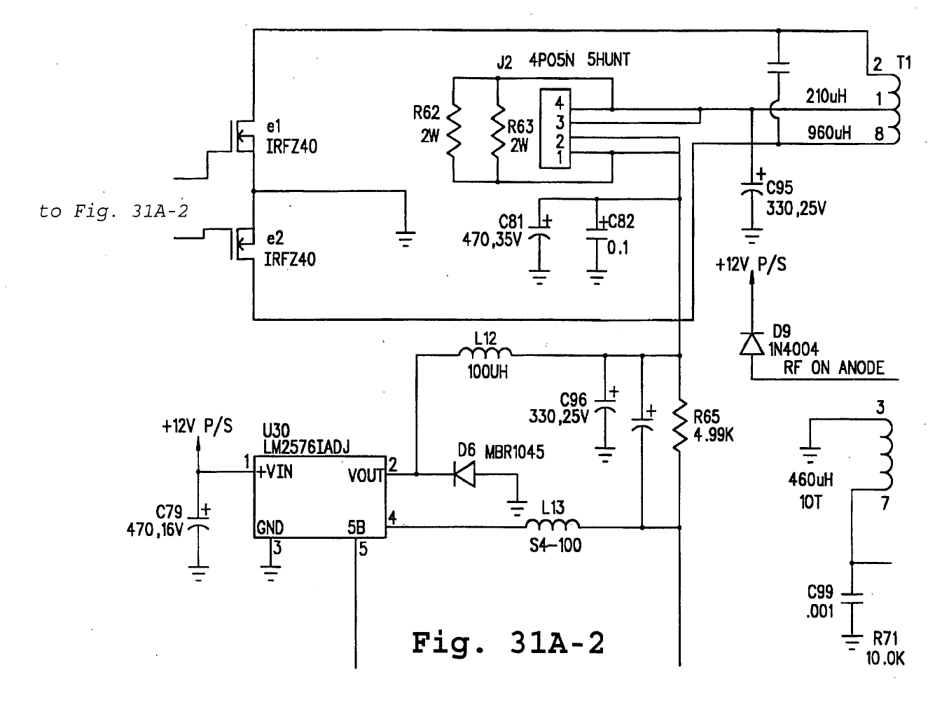

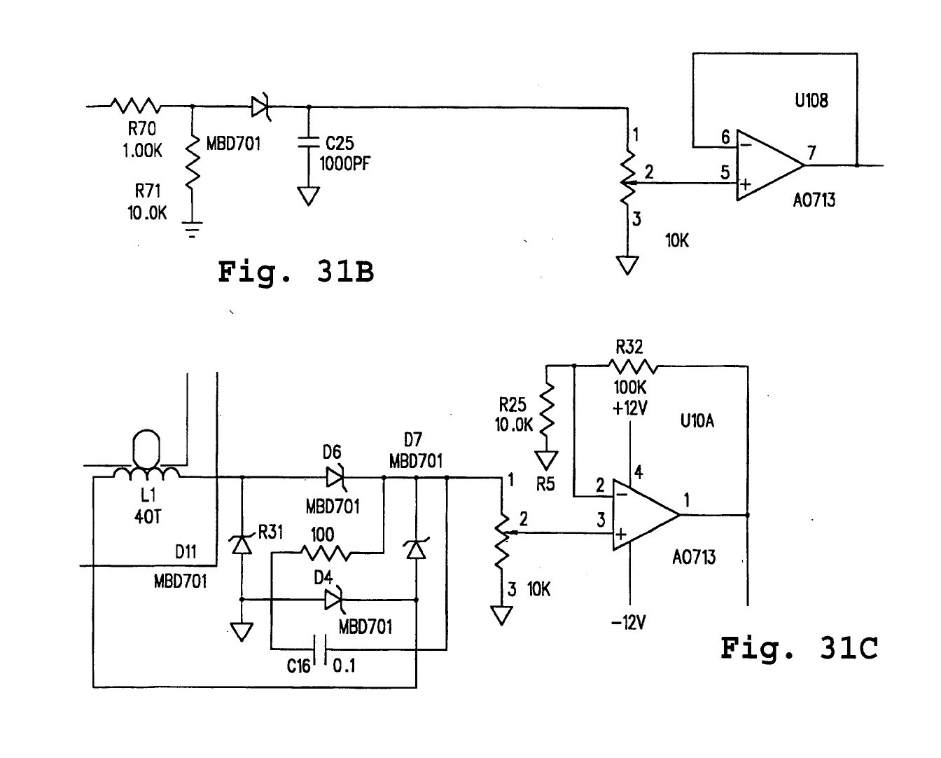

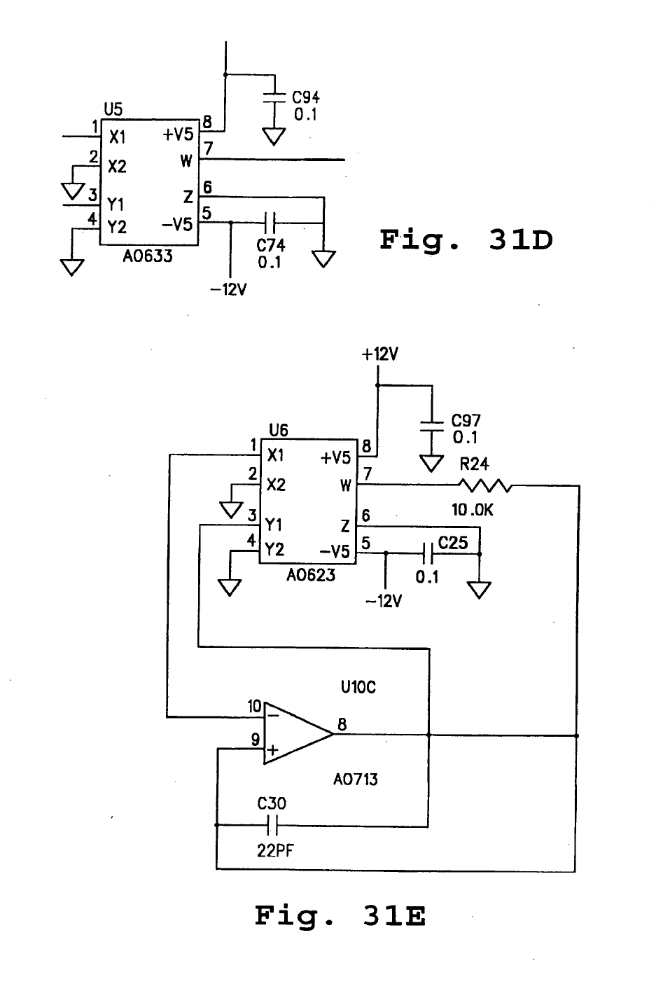

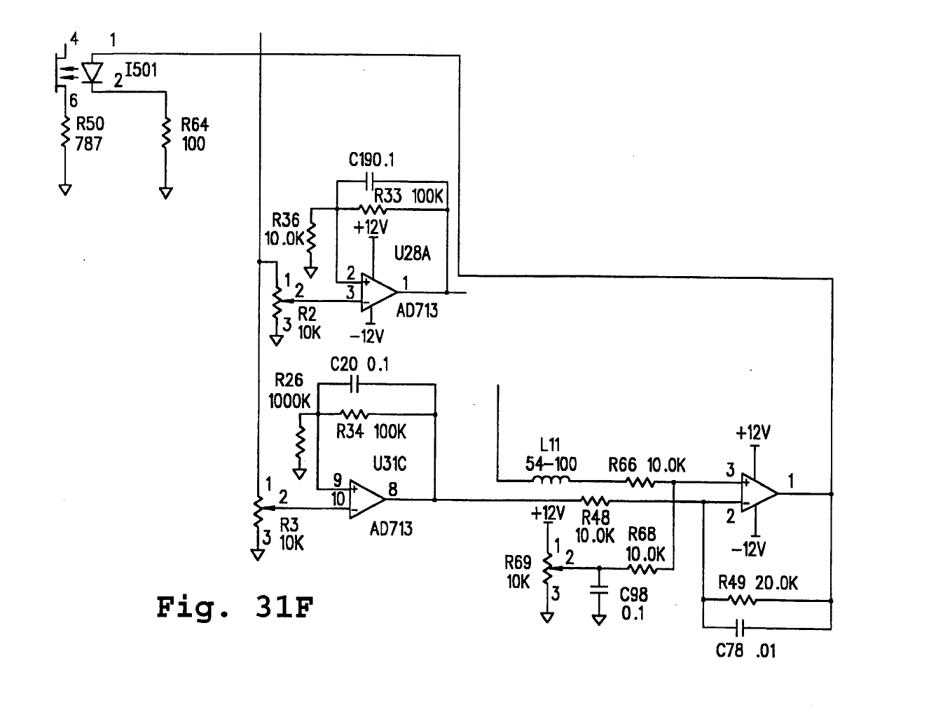

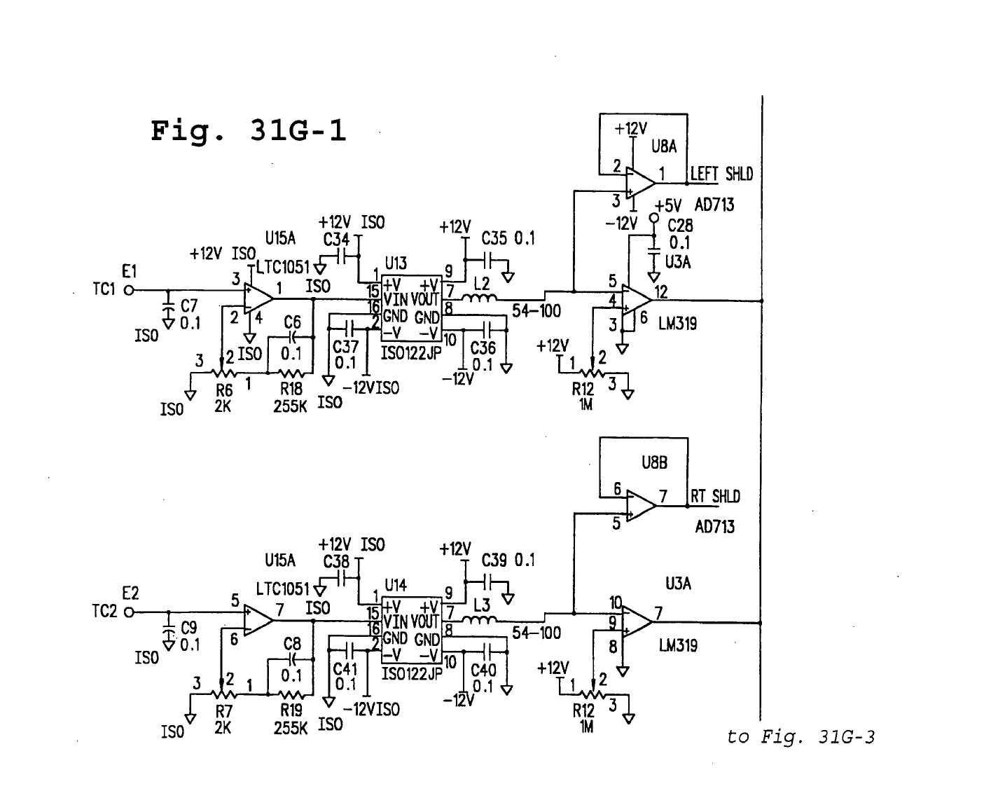

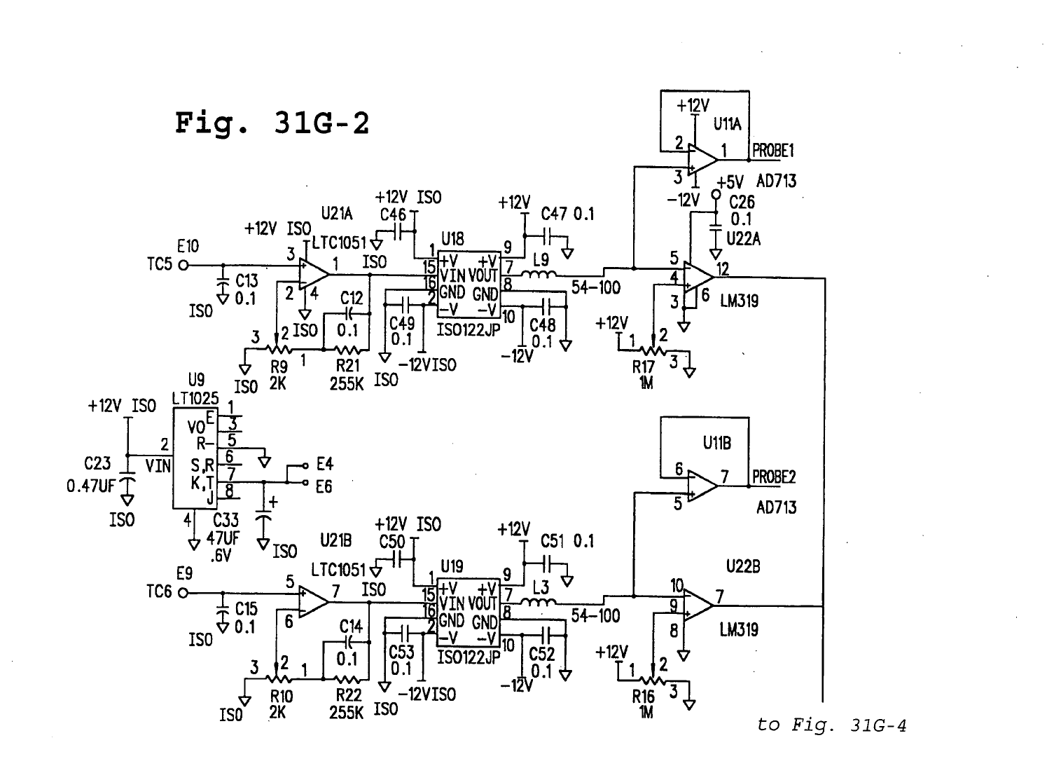

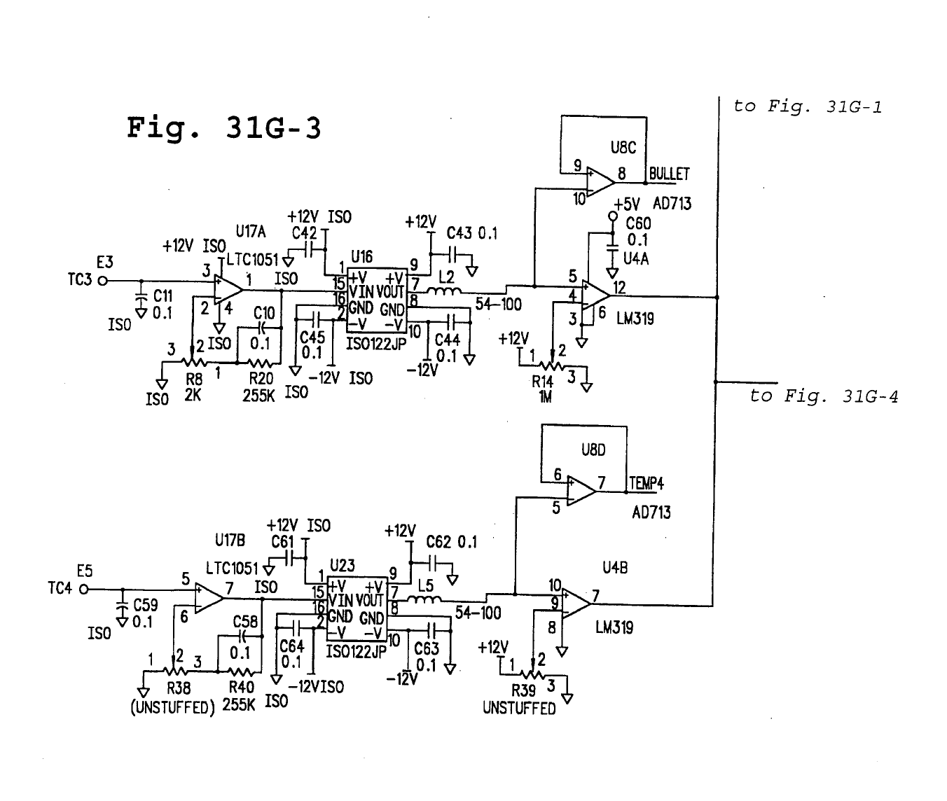

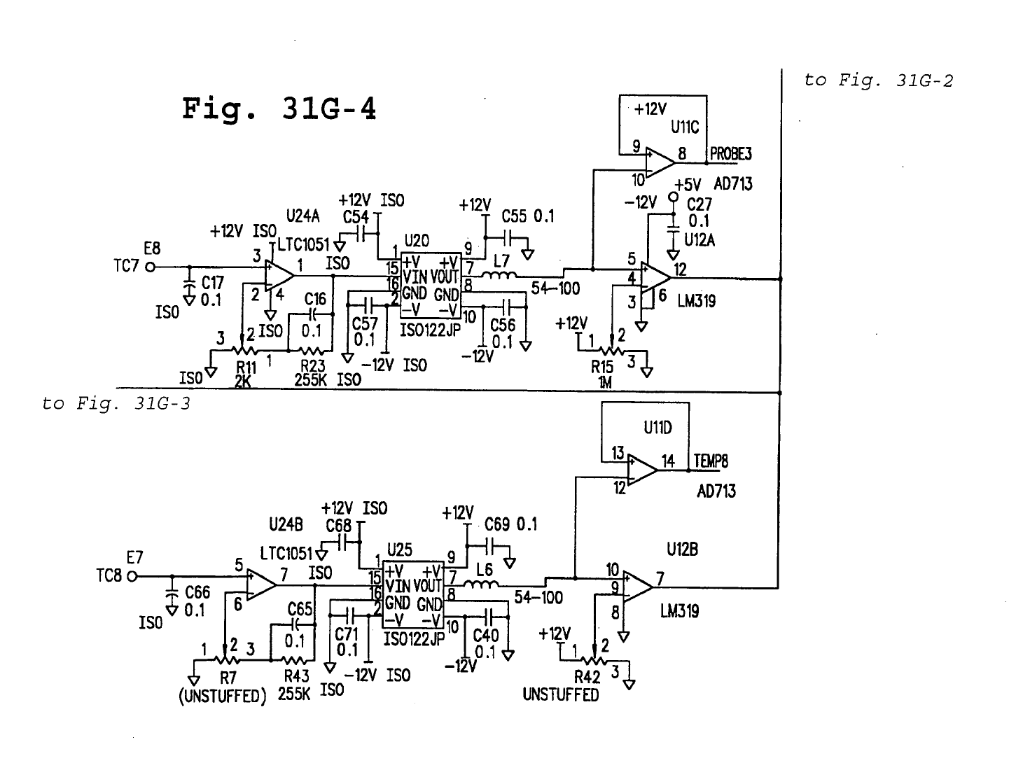

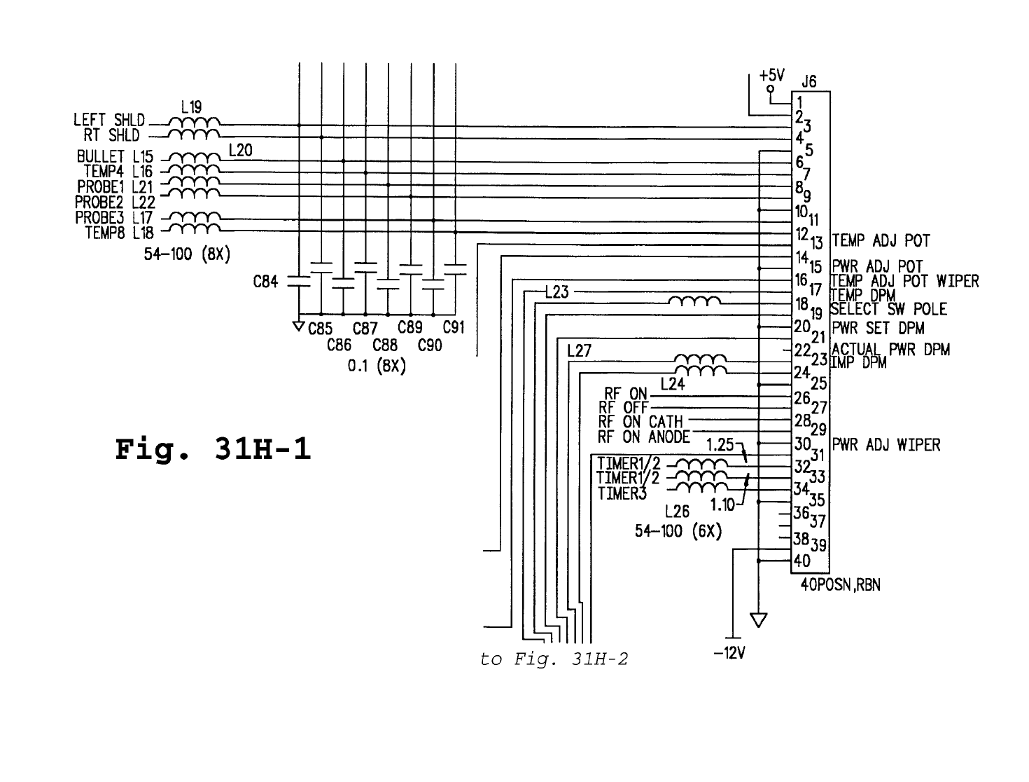

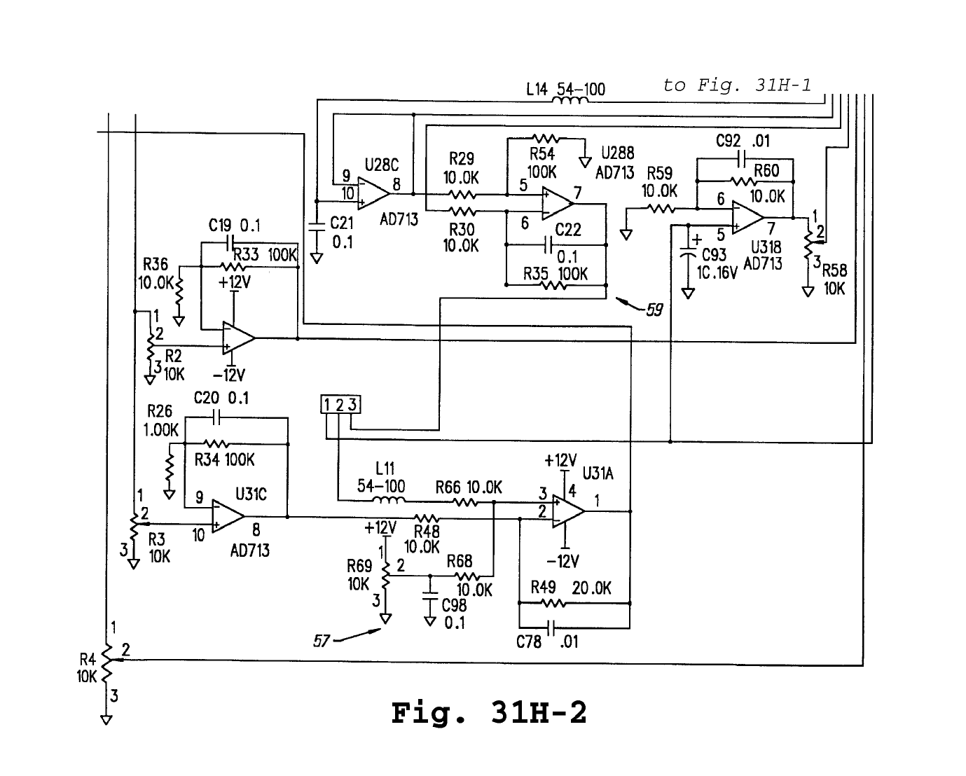

Referring now to FIG. 30, a block diagram of an energy source 152 is illustrated. Energy source 152 includes a power supply 154, power circuits 156, a controller 158, a power and impedance calculation device 160, a current sensor 162, a voltage sensor 164, a temperature measurement device 166 and a user interface and display 168. FIGS. 31A-1, 31A-2, 31B-31F and 31G-1 through 31G-4 are schematic diagrams of power supply 154, voltage sensor 164, current sensor 162, power computing circuit associated with power and impedance calculation device 160, impedance computing circuit associated with power and impedance calculation device 160, power control circuit of controller 158 and an eight-channel temperature measurement circuit of temperature measurement device 166, respectively.

Current delivered through each electrode 116 is measured by current sensor 162. Voltage between the electrodes 116 is measured by voltage sensor 164. Impedance and power are then calculated from the measured current and voltage at power and impedance calculation device 160. These values can then be displayed at user interface 168. Signals representative of power and impedance values are received by controller 158. A control signal is generated by controller 158 that is proportional to the difference between an actual measured value and a desired value. The control signal is used by power circuits 156 to adjust the energy output in an appropriate amount in order to maintain the desired energy delivered at the respective electrode 116. A profile of energy delivered can be incorporated in controller 158, and a pre-set amount of energy to be delivered can also be profiled.

In a similar manner, temperatures detected at sensors 124 and 126 provide feedback for maintaining a selected energy output. The actual temperatures are measured at temperature measurement device 166, and the temperatures are displayed at user interface 168. A control signal is generated by controller 159 that is proportional to the difference between an actual measured temperature and a desired temperature. The control signal is used by power circuits 157 to adjust the energy output in an appropriate amount in order to maintain the desired temperature detected at the respective sensor 124 or 126.

Circuitry, software and feedback to controller 158 result in process control, and are used to change: (i) the selected energy output, including RF, ultrasound and the like, (ii) the duty cycle (on-off and wattage), (iii) bipolar energy delivery, and (iv) fluid delivery (e.g., flow rate and pressure). These process variables are controlled and varied, while maintaining the desired delivery of energy output independent of changes in voltage or current, based on temperatures monitored at sensors 124 and 126 at multiple sites.

Controller 158 can be a digital or analog controller, or a computer with software. When controller 158 is a computer it can include a CPU coupled through a system bus. Controller 158 can include a keyboard, a disk drive, or other non-volatile memory systems, a display, and other peripherals, as are known in the art. Also coupled to the system bus are a program memory and a data memory. Controller 158 can be microprocessor controlled.

Referring now to FIG. 32, current sensor 162 and voltage sensor 164 are connected to the input of an analog amplifier 170. Analog amplifier 170 can be a conventional differential amplifier circuit for use with sensors 124 and 126. The output of analog amplifier 170 is sequentially connected by an analog multiplexer 172 to the input of analog-to-digital converter 174. The output of analog amplifier 170 is a voltage which represents the respective sensed temperatures. Digitized amplifier output voltages are supplied by analog-to-digital converter 174 to a microprocessor 176. Microprocessor 176 sequentially receives and stores digital representations of impedance and temperature. Each digital value received by microprocessor 176 corresponds to different temperatures and impedances. Microprocessor 176 may be a type 68HCII available from Motorola. However, it will be appreciated that any suitable microprocessor or general purpose digital or analog computer can be used to calculate impedance or temperature.

Calculated power and impedance values can be indicated on user interface 168. User interface 168 includes operator controls and a display. Alternatively, or in addition to the numerical indication of power or impedance, calculated impedance and power values can be compared by microprocessor 176 with power and impedance limits. When the values exceed predetermined power or impedance values, a warning can be given on interface 168, and additionally, the delivery of RF energy can be reduced, modified or interrupted. A control signal from microprocessor 176 can modify the power level supplied by power supply 154.

Controller 158 can be coupled to an imaging system. The imaging system can be used to perform diagnostics before, during and/or after treatment. For example, the imaging system may be used to first define the volume of the tumor or selected mass. Suitable imaging systems include but are not limited to, ultrasound, CT scanning, X-ray film, X-ray fluoroscope, magnetic resonance imaging, electromagnetic imaging and the like. The use of such devices to define a volume of a tissue mass or a tumor is well known to those skilled in the art.

Specifically with ultrasound, the image of a selected mass or tumor may be imported to user interface 168. The placement of electrodes 116 can be marked, and energy delivered to the selected site with prior treatment planning. Ultrasound can be used for real time imaging. Tissue characterization of the imaging can be utilized to determine how much of the tissue is heated. This process can be monitored. The amount of energy delivered is low, and the ablation or hyperthermia of the tissue is slow. Desiccation of tissue between the tissue and each needle 116 is minimized by operating at low power.

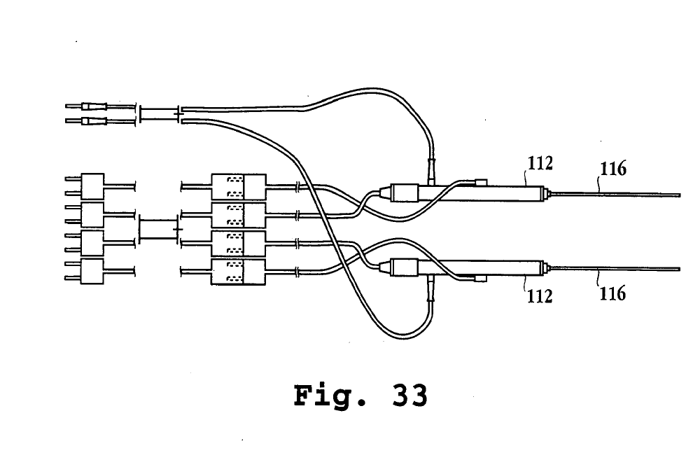

FIG. 34 illustrates another embodiment of the tissue treatment apparatus of the present application. Delivery catheter 12 has an insulation sleeve 28 disposed about a portion of its exterior. A distal portion 323 of delivery catheter 12 includes a conductive surface for energy delivery. Delivery catheter 12 is coupled to an energy source 40. Distal portions of electrodes 20 are deployed from distal end 16 of delivery catheter 12.

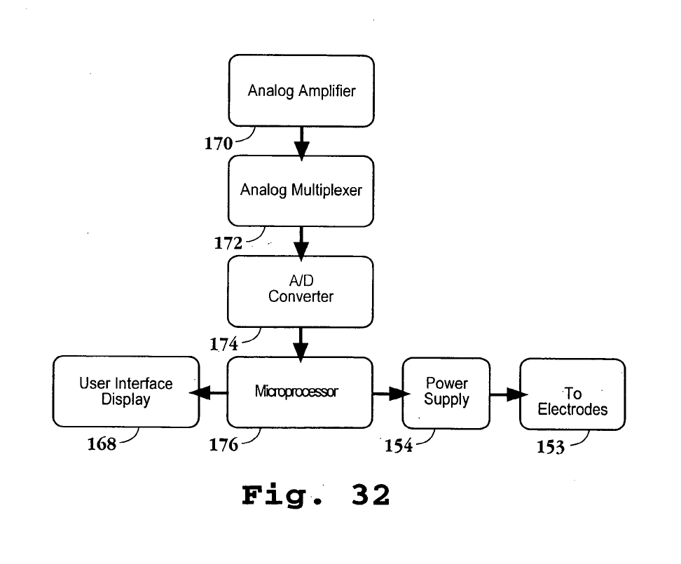

Examples listed in Table 1 below illustrate the use of multiple electrodes 116 to delivery RF energy in a bipolar mode to ablate tissue. Examples 1-12 use two treatment apparatuses with two electrodes 116 shown in FIG. 33, or a pair of RF electrodes 116. Examples 13-14 use two pairs of RF electrodes, or four RF electrodes.

TABLE 1

| | Exposed | Distance | | Abla- | |
|---|---|---|---|---|---|
| Exam- | Electrode | between | Power | tion | Lesion Size |
| ple | Length | Electrodes | Setting | Time | (W × L × D) |
| 1 | 1.5 cm | 1.5 cm | 5 W | 10 min | 2 × 1.7 × 1.5 cm³ |
| 2 | 1.5 cm | 2 cm | 7 W | 10 min | 2.8 × 2.5 × 2.2 cm³ |
| 3 | 2.5 cm | 2 cm | 10 W | 10 min | 3 × 2.7 × 1.7 cm³ |
| 4 | 2.5 cm | 2.5 cm | 8 W | 10 min | 2.8 × 2.7 × 3 cm³ |
| 5 | 2.5 cm | 2.5 cm | 8 W | 12 min | 2.8 × 2.8 × 2.5 cm³ |
| 6 | 2.5 cm | 1.5 cm | 8 W | 14 min | <3 × 3 × 2 cm³ |
| 7 | 2.5 cm | 2.5 cm | 8 W | 10 min | 3 × 3 × 3 cm³ |
| 8 | 2.5 cm | 2.5 cm | 10 W | 12 min | 3.5 × 3 × 2.3 cm³ |
| 9 | 2.5 cm | 2.5 cm | 11 W | 11 min | 3.5 × 3.5 × 2.5 cm³ |
| 10 | 3 cm | 3 cm | 11 W | 15 min | 4.3 × 3 × 2.2 cm³ |
| 11 | 3 cm | 2.5 cm | 11 W | 11 min | 4 × 3 × 2.2 cm³ |
| 12 | 4 cm | 2.5 cm | 11 W | 16 min | 3.5 × 4 × 2.8 cm³ |
| 13 | 2.5 cm | 2.5 cm | 12 W | 16 min | 3.5 × 3 × 4.5 cm³ |
| 14 | 2.5 cm | 2.5 cm | 15 W | 14 min | 4 × 3 × 5 cm³ |

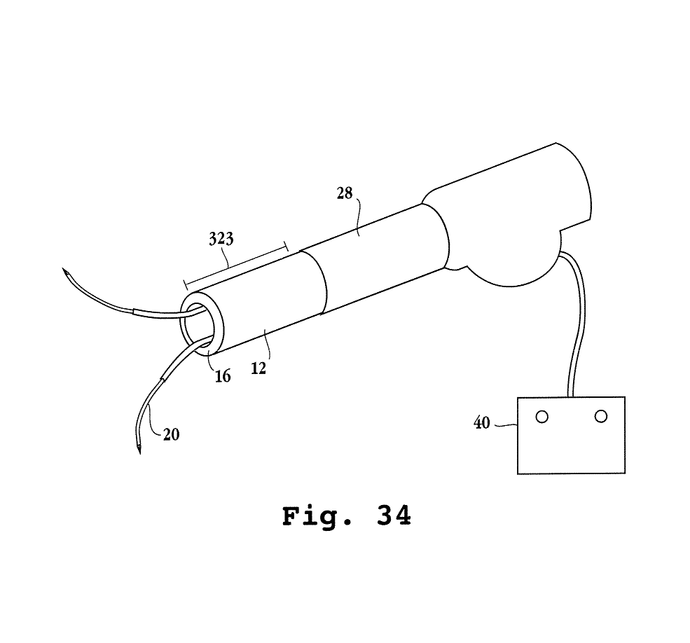

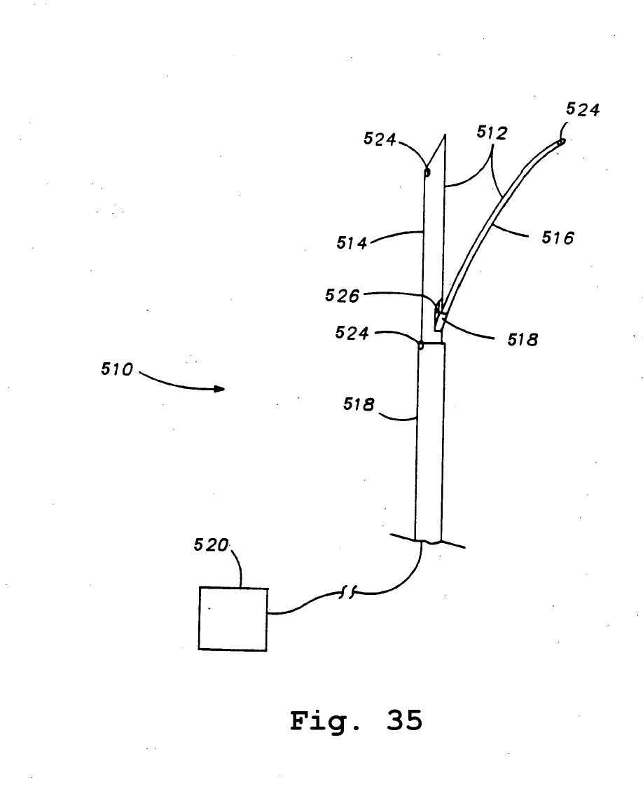

Referring now to FIG. 35, a treatment apparatus 510 includes a delivery device 512. Delivery device 512 includes a primary electrode 514 with an adjustable energy delivery surface (e.g., adjustable in length), and one or more secondary electrodes 516 that are typically introduced from a lumen formed at least partially in primary electrode 514. Each secondary electrode 516 also has an adjustable energy delivery surface (e.g., adjustable in length). The adjustability of the lengths of energy delivery surfaces permits ablation of a wide variety of geometric configurations of a targeted mass. Lengths of primary and secondary electrodes 514 and 516 are adjusted, and primary electrode 514 is moved up and down, rotationally about its longitudinal axis, and back and forth, in order to define, along with sensors, the periphery or boundary of the ablated mass and ablate a variety of different geometries that are not always symmetrical.

Primary electrode 514 is constructed so that it can be introduced in a percutaneous or laparoscopic manner into a solid mass. Primary electrode 514 can have a sharpened distal end 514′ to assist introduction into the solid mass. Each secondary electrode 516 is constructed to be less structurally rigid than primary electrode 514. This is achieved by: (i) choosing different materials for electrodes 514 and 516, (ii) using the same material but having less of it for secondary electrode 516, e.g., secondary electrode 516 is not as thick as primary electrode 514, or (ii) including another material in one of the electrodes 514 or 516 to vary their structural rigidity. For purposes of this application, structural rigidity is defined as the amount of deflection that an electrode has relative to its longitudinal axis. It will be appreciated that a given electrode will have different levels of rigidity depending on its length. Primary and secondary electrodes 514 and 516 can be made of a variety of conductive materials, both metallic and non-metallic. One suitable material is type 304 stainless steel of hypodermic quality. The rigidity of secondary electrode 516 can be about 10%, 25%, 50%, 75% and 90% of the rigidity of primary electrode 514. In some embodiments, secondary electrode 516 can be made of a shaped memory metal, such as NiTi, commercially available from Raychem Corporation, Menlo Park, Calif.

Each of primary or secondary electrode 514 or 516 can have different lengths. Suitable lengths for primary electrode 514 include but are not limited to 17.5 cm, 25.0 cm. and 30.0 cm. The actual length of an electrode depends on the location of the targeted solid mass to be ablated, its distance from the skin, its accessibility as well as whether or not the physician chooses a laparoscopic, percutaneous or other procedure. Further, treatment apparatus 510, and more particularly delivery device 12, can be introduced through a guide to the desired tissue mass site.

An insulation sleeve 518 is positioned around an exterior of one or both of the primary and secondary electrodes 514 and 516. Each insulation sleeve 518 may be adjustably positioned so that the lengths of energy delivery surfaces on each electrode can be varied. Each insulation sleeve 518 surrounding a primary electrode 514 can include one or more apertures, such as for the introduction of a secondary electrode 516 through primary electrode 514 and insulation sleeve 518. In one embodiment, insulation sleeve 518 can comprise a polyimide material, with a sensor positioned on top of the polyimide insulation (e.g., a 0.002-inch thick shrink wrap). The polyimide insulating layer is semi-rigid. The sensor can lay down substantially the entire length of the insulation.

An energy source 520 is connected with delivery device 512 with one or more cables 522. Energy source 520 can be an RF energy source, a microwave source, a short wave source, a laser source and the like. Delivery device 512 can be comprised of primary and secondary electrodes 514 and 516 that are RF electrodes, microwave antennas, as well as combinations thereof. Energy source 520 may be a combination RF/microwave box. Further a laser optical fiber, coupled to a laser source 520 can be introduced through one or both of primary or secondary electrodes 514 and 516. One or more of the primary or secondary electrodes 514 and 516 can be an arm for the purposes of introducing the optical fiber.

One or more sensors 524 are positioned on interior or exterior surfaces of primary electrode 514, secondary electrode 516 or insulation sleeve 518. Sensors 524 may be positioned at primary electrode distal end 514′, secondary electrode distal end 516′ and insulation sleeve distal end 518′. Sensors 524 may be thermal sensors that permit accurate measurement of temperature at a tissue site in order to determine: (i) the extent of ablation, (ii) the amount of ablation, (iii) whether or not further ablation is needed, and (iv) the boundary or periphery of the ablated mass. Further, sensors 524 prevent non-targeted tissue from being destroyed or ablated.

Sensors 524 are of conventional design, including but not limited to thermistors, thermocouples, resistive wires, and the like. Suitable thermal sensors 524 include a T type thermocouple with copper-constantan, J type thermocouples, E type thermocouples, K type thermocouples, fiber optics, resistive wires, thermocouple IR detectors, and the like. It will be appreciated that sensors 524 need not be thermal sensors.

Sensors 524 measure temperature and/or impedance to permit monitoring ablation so that a desired level of ablation is achieved without destroying too much healthy tissue. This reduces damage to healthy tissue surrounding the targeted mass to be ablated. By monitoring the temperature at various points within the interior of the selected mass, a determination of the tumor periphery can be made, as well as a determination of when ablation is complete. If at any time sensor 524 determines that a desired ablation temperature is exceeded, then an appropriate feedback signal is received at energy source 520 which then regulates the amount of energy delivered to primary and/or secondary electrodes 514 and 516. Thus the geometry of the ablated mass is selectable and controllable. Any number of different ablation geometries can be achieved.

Secondary electrode 516 is laterally deployed out of an aperture 526 formed in primary electrode 514. Aperture 526 may be positioned along the longitudinal axis of primary electrode 514. Initially, primary electrode 514 is introduced into or adjacent to a target solid mass. Secondary electrode 516 is then introduced out of aperture 526 into the solid mass. There is wide variation in the mount of deflection of secondary electrode 516. For example, secondary electrode 516 can be deflected a few degrees from the longitudinal axis of primary electrode 514, or secondary electrode 516 can be deflected in any number of geometric configurations, including but not limited to a “J” hook. Further, secondary electrode 516 is capable of being introduced from primary electrode 514 to a few millimeters away from primary electrode 514, or a much larger distance from primary electrode 514. Ablation by secondary electrode 516 can begin a few millimeters away from primary electrode 514, or when secondary electrode 516 is advanced a greater distance from primary electrode 514.

Referring now to FIG. 36, primary electrode 514 has been introduced into a tumor 528, or other solid mass. After primary electrode 514 has been introduced, secondary electrode 516 is advanced out of aperture 526 and deployed within tumor 528. Insulation sleeves 518 are adjusted for primary and secondary electrodes 514 and 516. Energy (e.g., RF) is delivery through electrode 516 to tumor 528 in a monopolar mode, or alternatively, between multiple electrodes 516 and 514 in a bipolar mode. Delivery device 512 can be switched between monopolar and bipolar operations and has multiplexing capability between electrodes 514 and 516. In the bipolar mode, ablation may occur between secondary electrode 516 and primary electrode 514. Secondary electrode 516 is retracted back into primary electrode 514. Primary electrode 514 is then rotated. Secondary electrode 516 is then re-deployed within tumor 528. Secondary electrode 516 may be introduced a short distance within tumor 528 to ablate a small area. It can then be advanced further within tumor 528 any number of times to create more ablation zones. Secondary electrode 516 is retracted back into primary electrode 514. Primary electrode 514 can be: (i) rotated again, (ii) moved along a longitudinal axis of tumor 528 to begin another series of ablations with secondary electrode 516 being deployed and retracted, or (iii) removed from tumor 528. Parameters permitting ablation of tumors and masses of different sizes and shapes (including a series of ablations) include: the use of primary and secondary electrodes 514 and 516 with variable energy delivery surfaces, and the use of sensors 524.

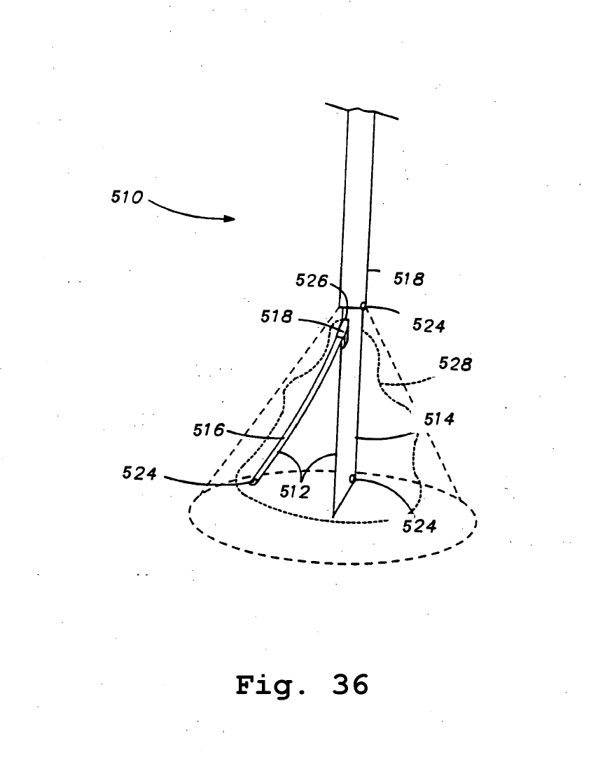

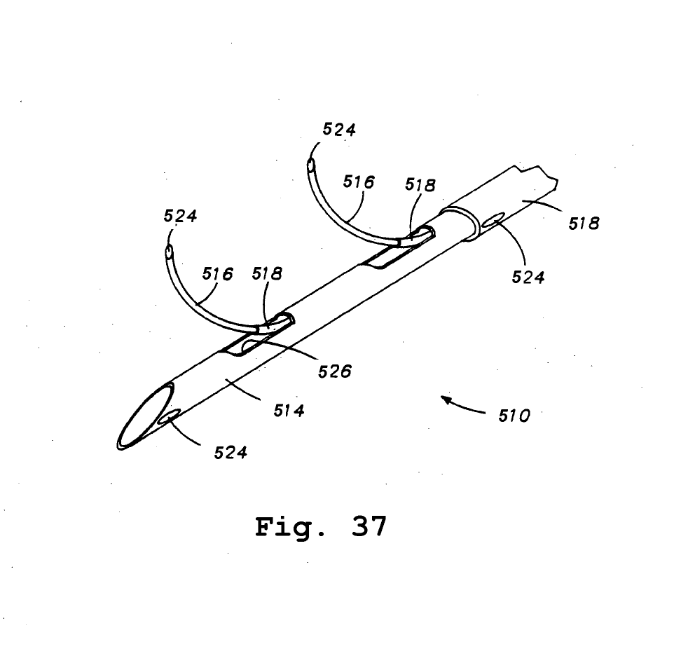

As illustrated in FIG. 37, treatment device 510 can include two or more secondary electrodes 516 which can be independently or dependently laterally deployed along different positions along the longitudinal axis of primary electrode 514. Each secondary electrode 516 is advanced out of a separate aperture 526 formed in the body of primary electrode 514. Multiple secondary electrodes 516 can all be introduced along the same planes, a plurality of planes or a combination of both.

Primary electrode 514 can be introduced in an adjacent relationship to tumor 528, as illustrated in FIG. 38. As shown, two secondary electrodes 516 are deployed from primary electrode 514 at opposite ends of irregularly shaped tumor 528. Operating in the bipolar mode, an ablation area is defined between the two secondary electrodes 516. This deployment is useful for small tumors, or where piercing tumor 528 is not desirable. Primary electrode 514 can be rotated, with secondary electrodes 516 retracted into a central lumen of primary electrode 514. After secondary electrodes 516 are re-deployed, another ablation volume defined between the two secondary electrodes 516 may be created. Further, primary electrode 514 can be withdrawn from its initial adjacent position to tumor 528, repositioned to another position adjacent to tumor 528, and secondary electrodes 516 re-deployed to begin another ablation cycle. Any variety of different electrode position patterns may be utilized to create desired ablations for tumors of different geometries and sizes.

In FIG. 39, a center of tumor 528 is pierced by primary electrode 514, secondary electrodes 516 are laterally deployed and retracted (between which an ablation in tumor 528 is made), primary electrode 14 is rotated, secondary electrodes 516 are deployed and retracted (between which another ablation in tumor 528 is made), and so on until a cylindrical ablation volume is achieved. Delivery device 512 can be operated in the bipolar mode, such as between the two secondary electrodes 516, or between a secondary electrode 516 and primary electrode 514. Alternatively, delivery device 512 can be operated in a monopolar mode.

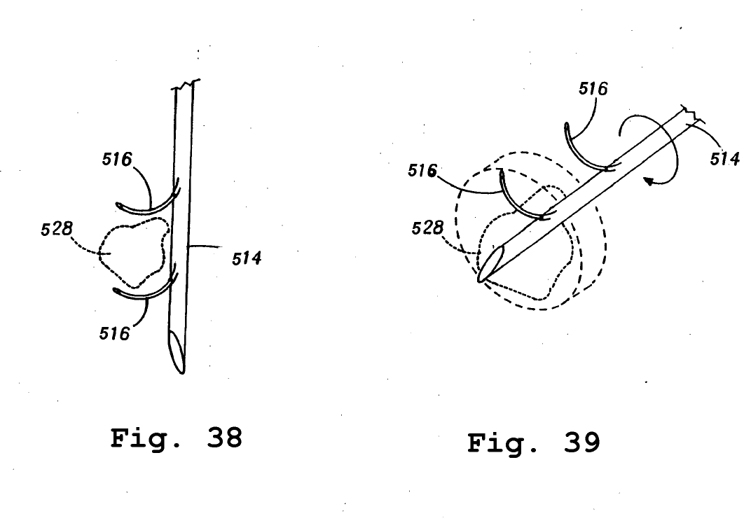

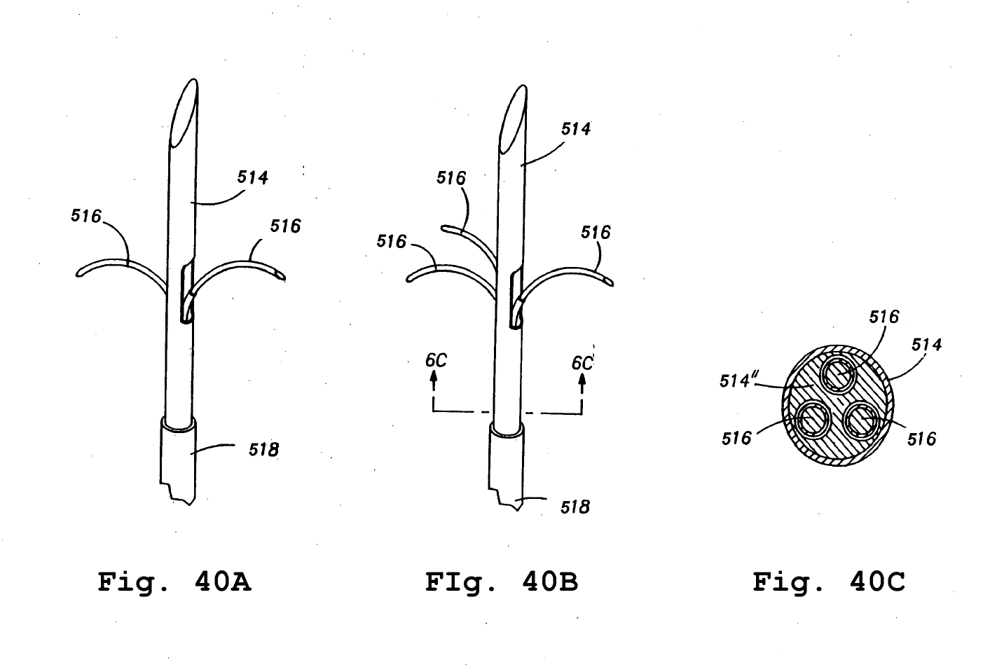

Secondary electrodes 516 can serve the additional function of anchoring delivery device 512 in a selected mass, as illustrated in FIGS. 40A and 40B. In FIG. 40A, one or both secondary electrodes 516 are used to anchor and position primary electrode 514. Further, one or both secondary electrodes 516 are also used to ablate tissue. In FIG. 40B, three secondary electrodes 516 are deployed to anchor primary electrode 514.

FIG. 40C illustrates the infusion capability of delivery device 512. Three secondary electrodes 516 are positioned in a central lumen 514″ of primary electrode 514. One or more of the secondary electrodes 516 can also include in each a central lumen coupled to an infusion source (not shown). Central lumen 514″ is coupled to an infusion source (not shown) and delivers a variety of fluidic mediums to selected places both within and outside of the targeted ablation mass. Suitable fluidic mediums include but are not limited to, therapeutic agents, conductivity enhancement mediums, contrast agents or dyes, and the like. An example of a therapeutic agent is a chemotherapeutic agent.

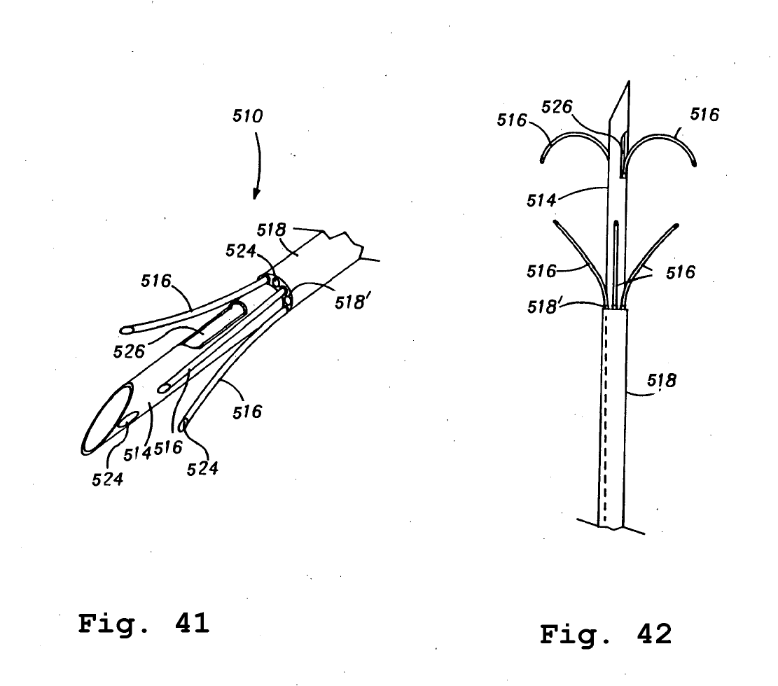

As shown in FIG. 41, insulation sleeve 518 can include one or more lumens for receiving secondary electrodes 516 which are deployed out of an insulation sleeve distal end 518′. FIG. 42 illustrates three secondary electrodes 516 being introduced out of insulation sleeve distal end 518′, and two secondary electrodes 516 introduced through apertures 526 formed in primary electrode 514. As illustrated, secondary electrodes 516 deployed through apertures 526 provide an anchoring function. It will be appreciated that FIG. 42 shows that secondary electrodes 516 can have a variety of different geometric configurations in delivery device 512.

Resources, which may include hardware, software, or a combination of both, are connected with sensors 524, primary and secondary electrodes 514 and 516 and energy source 520 to control delivery and maintenance of selected energy output at primary and secondary electrodes 514 and 516 (e.g., feedback control), including energy output maintenance for a selected length of time. It will be appreciated that devices similar to those associated with RF energy can be utilized with laser optical fibers, microwave devices, and the like.

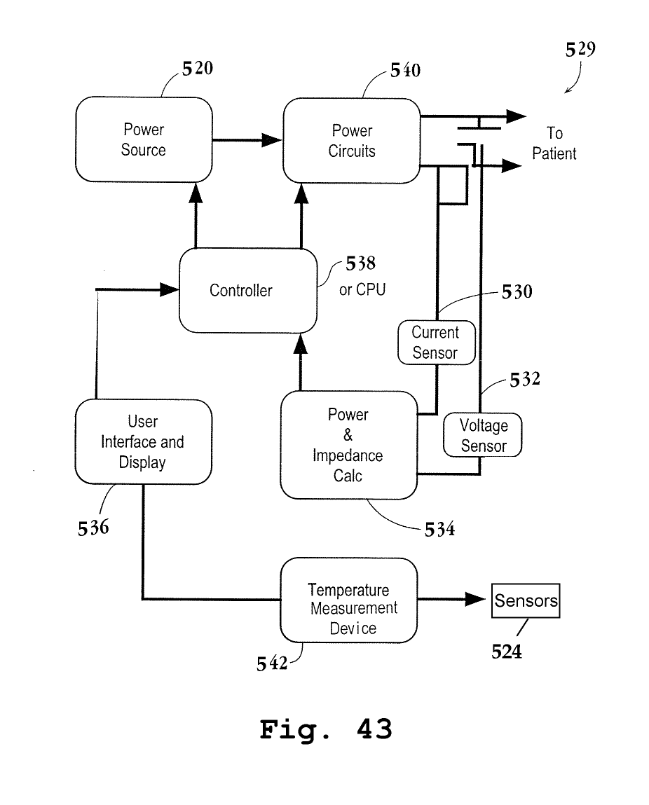

Referring now to the control system 529 illustrated in FIG. 43, current delivered through primary and secondary electrodes 514 and 516 is measured by current sensor 530. Voltage is measured by voltage sensor 532. Impedance and power are then calculated at power and impedance calculation device 534. These values can then be displayed at user interface and display 536. Signals representative of power and impedance values are received by controller 538. Calculated power and impedance values can be indicated on user interface and display 536. User interface and display 536 can further include operator controls and a display. A control signal is generated by controller 538 that is proportional to the difference between an actual measured value and a desired value. The control signal is used by power circuits 540 to adjust the energy output in an appropriate amount in order to maintain the desired energy delivered at the respective primary and/or secondary electrodes 514 and 516. A profile of energy delivered can be incorporated in controller 538. A preset amount of energy to be delivered can also be profiled.

In a similar manner, temperatures detected at sensors 524 provide feedback for control and maintenance of a selected energy output. The actual temperatures are measured at temperature measurement device 542, and the temperatures are displayed at user interface and display 536. A control signal is generated by controller 538 that is proportional to the difference between an actual measured temperature and a desired temperature. The control signal is used by power circuits 540 to adjust the energy output in an appropriate amount in order to reach or maintain the desired temperature detected at the respective sensors 524. A multiplexer can be included to measure current, voltage and temperature, at the numerous electrodes 514 and 516 and sensors 524.

Circuitry, software and feedback to controller 538 result in process control, and the maintenance of the selected power that is independent of changes in voltage or current, and are used to change: (i) the selected power, including RF, microwave, laser and the like, (ii) the duty cycle (on-off and wattage), (iii) bipolar or monopolar energy delivery, and (iv) infusion medium delivery, including flow rate and pressure. These process variables are controlled and varied, while maintaining the desired delivery of energy independent of changes in voltage or current, based on temperatures monitored at sensors 524.

Controller 538 can be coupled to imaging systems, including but not limited to ultrasound, CT scanners, X-ray, MRI, mammographic X-ray and the like. Further, direct visualization and tactile imaging can be utilized. Controller 538 can be a digital or analog controller, or a computer with software. When controller 538 is a computer it can include a CPU coupled through a system bus. On this system can be a keyboard, a disk drive, or other non-volatile memory systems, a display, and other peripherals, as are known in the art. Also coupled to the bus are a program memory and a data memory.

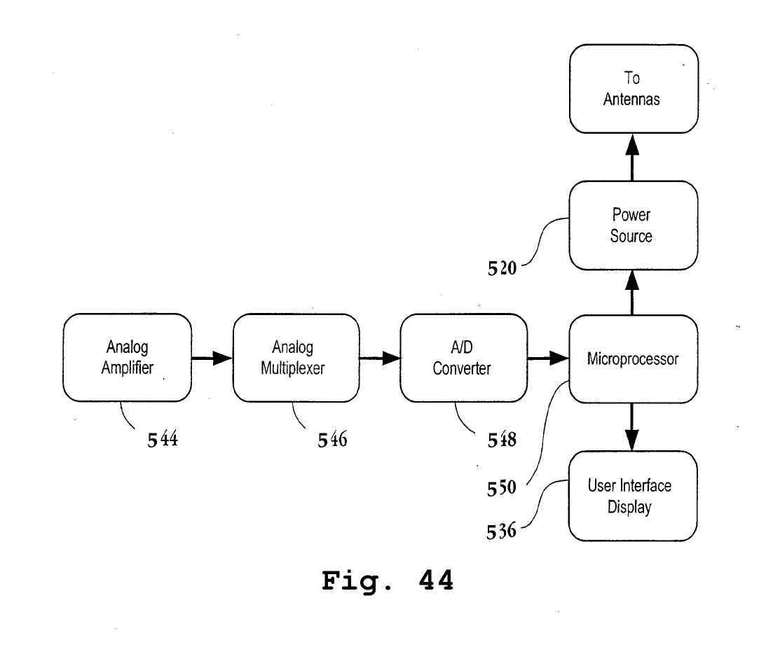

Referring now to FIGS. 43 and 44, current sensor 530 and voltage sensor 532 are connected to the input of an analog amplifier 544. Analog amplifier 544 can be a conventional differential amplifier circuit for use with sensors 524. The output of analog amplifier 544 is sequentially connected by an analog multiplexers 46 to the input of A/D converter 548. The output of analog amplifier 544 is a voltage which represents the respective sensed temperatures. Digitized amplifier output voltages are supplied by A/D converter 548 to a microprocessor 550.

Microprocessor 550 may be Model No. 68HCII available from Motorola. However, it will be appreciated that any suitable microprocessor or general purpose digital or analog computer can be used to calculate impedance or temperature. Microprocessor 550 sequentially receives and stores digital representations of impedance and temperature. Each digital value received by microprocessor 550 corresponds to different temperatures and impedances. Alternatively, or in addition to the numerical indication of power or impedance, calculated impedance and power values can be compared by microprocessor 550 with power and impedance limits. When the values exceed predetermined power or impedance values, a warning can be given on user interface and display 536, and additionally, the delivery of energy can be reduced, modified or interrupted. A control signal from microprocessor 550 can modify the power level supplied by power source 520.

The foregoing description of preferred embodiments of the present application has been provided for the purposes of illustration and description. It is not intended to be exhaustive or to limit the invention to the precise forms disclosed. Obviously, many modifications, variations and different combinations of embodiments will be apparent to practitioners skilled in this art. Also, it will be apparent to the skilled practitioner that elements from one embodiment can be recombined with one or more other embodiments. It is intended that the scope be defined by the following claims and their equivalents.

1. A delivery device, comprising:
a needle; and
at least one deployable electrode retractable into the needle in a retracted geometry that is substantially straight,
wherein the at least one deployable electrode is operatively connectable to a radiofrequency energy source for delivery of radiofrequency energy, at least a distal portion of the at least one electrode is deployable, from the needle in a lateral direction relative to a longitudinal axis of the needle, to a deployed geometry that comprises at least one radius of curvature in three planes.

2. The delivery device of claim 1, wherein the deployed geometry comprises a helical portion.

3. The delivery device of claim 1, wherein the needle comprises an insulation sleeve and an exposed distal portion.

4. The delivery device of claim 1, wherein a distal portion of the needle comprises a thermal sensor.

5. The delivery device of claim 1, wherein the at least one electrode comprises three electrodes, each comprising at least one radius of curvature in three planes.

6. The delivery device of claim 1, wherein the distal portion of the at least one electrode is deployable from a distal end of the needle.

7. A delivery device, comprising:
a skin puncturing means; and
at least one electrode retractable into the puncturing means in a retracted geometry,
wherein the at least one electrode is operatively connectable to a radiofrequency energy source, at least a distal portion of the at least one electrode is deployable from the puncturing means to a deployed geometry that comprises at least one radius of curvature in two or more planes.

8. The delivery device of claim 7, wherein the distal portion of the at least one electrode is deployable from the puncturing means in a lateral direction relative to a longitudinal axis of the puncturing means.

9. The delivery device of claim 7, wherein the distal portion of the at least one electrode is deployable from a distal end of, or a side opening along, the puncturing means.

10. The delivery device of claim 7, wherein the deployed geometry comprises a helical portion.

11. The delivery device of claim 7, wherein the puncturing means is selected from the group consisting of an insert, an introducer, a needle, and an electrode.

12. The delivery device of claim 7, further comprising a handle coupled to the puncturing means.

13. The delivery device of claim 7, wherein the puncturing means comprises an insulation sleeve and an exposed distal portion.

14. The delivery device of claim 7, wherein the retracted geometry is substantially straight.

15. The delivery device of claim 7, wherein a distal portion of the puncturing means comprises a thermal sensor.

16. The delivery device of claim 7, wherein a distal portion of the puncturing means comprises a thermocouple.

17. A method of delivery, comprising:
providing a delivery device that comprises a skin puncturing means and at least one electrode retractable in a substantially straight geometry within the puncturing means, wherein the at least one electrode is operatively connectable to a radiofrequency energy source; and
deploying at least a distal portion of the at least one electrode from the puncturing means so that the deployed distal portion comprises at least one radius of curvature in two or more planes.

18. The method of claim 17, further comprising:
deploying the distal portion of the at least one electrode in a lateral direction relative to a longitudinal axis of the puncturing means.

19. The method of claim 17, further comprising:
providing a thermal sensor on a distal portion of the puncturing means for measuring tissue temperature.
